(19) United States
(12) Patent Application Publication
(10) Pub. No.: US 2021/0188953 A1
(43) Pub. Date: Jun. 24, 2021

(54) HETEROMULTIMERIC BINDING MOLECULES FOR TOXIN NEUTRALIZATION, COMPOSITIONS AND METHODS THEREOF

(71) Applicant: Trustees of Tufts College, Medford, MA (US)

(72) Inventor: Charles B. Shoemaker, Medford, MA (US)

(73) Assignee: Trustees of Tufts College, Medford, MA (US)

(21) Appl. No.: 16/761,969

(22) PCT Filed: Sep. 6, 2018
(86) PCT No.: PCT/US2018/049695

§ 371 Date: May 6, 2020

Related U.S. Application Data
(60) Provisional application No. 62/582,596, filed on Nov. 7, 2017.

(51) Int. Cl. C07K 16/12; A61K 9/51; A61P 31/04

(52) U.S. Cl. CPC C07K 16/1282; A61K 9/5123; A61P 31/04; A61K 2039/505; C07K 2317/569; C07K 2317/76; C07K 2317/31

(57) ABSTRACT

Provided are multicomponent heteromultimeric binding protein molecules, which comprise heavy-chain-only variable domains (VHH) that specifically bind and neutralize target disease-causing agents, e.g., toxin proteins, such as C. difficile toxin A and/or toxin B, or C. botulinum toxin A and/or toxin B. The multimeric or heteromultimeric VHH-based neutralizing agents are called VNA binding molecules. Also provided are polynucleotides which encode the VNA binding molecules. Compositions, methods and kits comprising such multimeric binding protein molecules and polynucleotides are provided for treating a subject who has been exposed to, or who is at risk of exposure to, such disease-causing agents.

### STATEMENT OF RIGHTS TO INVENTIONS MADE UNDER FEDERALLY SPONSORED RESEARCH

This invention was made with government support under grant HR0011-14-2-0005 awarded by the Department of Defense and grants AI057159, AI030050, AI088748, DK084509, and AI099458 awarded by the National Institutes of Health. The government has certain rights in the invention.

### BACKGROUND OF THE INVENTION

High affinity binding agents, other than classical antibodies, that neutralize disease causing agents for the treatment of both routine incidents of disease and pandemics are needed worldwide, particularly to combat infection and intoxication of subjects by a variety of pathogenic and toxigenic microorganisms.

The production and storage of classical antibodies involve labor-intensive and costly processes. In fact, development of a single antibody therapeutic agent often requires years of clinical study and testing. Often, multiple, different therapeutic antibodies are necessary for the effective treatment of patients exposed to a disease agent, an outbreak of infection, or a bioterror assault. Developing and producing multiple antibodies that can bind to different targets (e.g. microbial pathogens, viral pathogens, toxins, and cancer cells) are difficult to achieve, as multiple antibodies for each pathogen or toxin need to be separately produced, stored and transported. Producing and stockpiling sufficient amounts of antibodies to protect and treat large populations present challenges, which have not been met to date. In addition, the shelf-life and stability of stored antibodies are often relatively short (e.g., weeks or months), thus requiring the production of new batches of antibodies to replace those that have expired due to degradation or other adverse effects on the integrity of antibody proteins.

Accordingly, there is a need for cost effective alternative therapeutic agents for treating disease-causing agents, including microorganisms and tumor cells, as well as for treating subjects afflicted with the diseases resulting from these foreign and unwelcome agents. A need also exists for alternative therapeutics that are easier to develop and produce and have a longer shelf life for use against disease-causing pathogens of various types, in particular, Clostridium species (spp.), such as Clostridium difficile. The present invention provides a solution to these needs.

### SUMMARY OF THE INVENTION

The present invention generally features heteromultimeric binding protein molecules (also called heteromultimeric binding molecules herein) that comprise modular components and are designed to bind specifically to two or more target disease agents or proteins, e.g., toxin proteins, which cause disease, toxicity, pathogenesis, and ill health in a subject. Described herein are heteromultimeric binding molecules that specifically neutralize the two or more target agents (e.g., toxin proteins); polynucleotides encoding the binding molecules; compositions comprising the binding molecules (or their encoding polynucleotides); methods of using the binding molecules; and kits containing the binding molecules. Following binding and neutralization of the target disease agent or toxin protein, the binding molecules may be eliminated from a cell and cleared from the treated subject, in particular, by the co-administration of antibodies that specifically bind to specific epitopes contained in the binding molecule.

The heteromultimeric binding molecules described herein are single agent therapeutics that have widespread application in the treatment of diseases and pathologies resulting from infection by microorganisms (e.g., pathogenic bacteria), in particular, Clostridium difficile or Clostridium botulinum bacteria. In embodiments, the binding molecules described herein can bind bispecifically or tetraspsecifically to target sites on the same or different disease-causing molecules or toxin proteins. The binding molecules of the invention are particularly useful for binding, neutralizing and eradicating disease-causing targets, such as toxins (toxin proteins and peptides) produced by microorganisms that intoxicate their host and cause debilitating disease, and even death. In embodiments, the invention encompasses polynucleotides (nucleic acid sequences) that encode the operably linked modular components that constitute the binding molecules described herein. In embodiments, the binding molecules are the proteins (polypeptides) encoded by the polynucleotides. In embodiments, the polynucleotide is DNA, cDNA, RNA, mRNA, and the like.

In one of its aspects, the present invention provides a polynucleotide encoding a heteromultimeric binding molecule comprising modular components which specifically target and bind to one or more toxin proteins produced by the bacterium Clostridium difficile (C. difficile). In an embodiment, the polynucleotide encodes a binding molecule that is bispecific for neutralizing a disease-causing agent such as a toxin produced by a pathogen. By way of example, the polynucleotide encodes a binding molecule that comprises two separate binding components, each of which specifically targets and binds to the same or different binding sites on a toxin of C. difficile. In particular embodiments, the toxin is toxin A (TcdA) and/or toxin B (TcdB) of C. difficile. In another aspect, the polynucleotide encodes a binding molecule that comprises two separate binding components, each of which specifically targets and binds to the same or different binding sites on a toxin (neurotoxin) produced by the bacterium Clostridium botulinum (called Botulinum herein), such as Botulinum toxin of serotype A (i.e., Botulinum toxin A).

In an embodiment, the polynucleotide encodes a binding molecule that comprises two separate binding components, each of which specifically targets and binds to binding sites on toxin A or to binding sites on toxin B of C. difficile. In another embodiment, the polynucleotide encodes a binding molecule that comprises two separate binding components, each of which specifically targets and binds to a different or non-overlapping binding site on toxin A or on toxin B of C. difficile. In another embodiment, the polynucleotide encodes a binding molecule that comprises two separate binding components, one of which specifically targets and binds to toxin A of C. difficile and the other of which specifically targets and binds to toxin B of C. difficile. In a particular embodiment, the binding component of the binding molecule is a heavy-chain-only variable H domain (VHH) molecule generated by the Camelidae species, which specifically binds toxin A or toxin B of C. difficile.

In another particular embodiment, the polynucleotide encodes a neutralizing binding molecule that comprises two separate binding components, each of which specifically targets and binds to a different (non-overlapping) binding site on Botulinum toxin A or toxin B. In another embodiment, the polynucleotide encodes a neutralizing binding molecule that comprises two separate binding components, each of which specifically targets and binds to a different (non-overlapping) binding site on Botulinum toxin A or on Botulinum toxin B. In another embodiment, the polynucleotide encodes a neutralizing binding molecule that comprises two separate binding components, one of which specifically targets and binds to Botulinum toxin A and the other of which specifically targets and binds to Botulinum toxin B. In a particular embodiment, the neutralizing binding components of the binding molecule are heavy chain variable domains of camelid heavy chain-only antibodies (VHHs), which specifically bind toxin A or toxin B of Botulinum. In an embodiment of any of the above, the polynucleotide is a deoxyribonucleic acid (DNA). In an embodiment of any of the above, the polynucleotide is a ribonucleic acid (RNA). In a particular embodiment of any of the above, the polynucleotide is messenger RNA (mRNA).

In another embodiment, the polynucleotide encodes a binding molecule that is tetraspecific for binding and neutralizing disease-causing toxin proteins produced by one or more bacterial pathogens. By way of specific example, the polynucleotide encodes a tetraspecific, neutralizing binding molecule that comprises four separate binding components, two of which specifically target and bind to different sites on one toxin, and two of which specifically target and bind to the different sites on a second toxin. In embodiments, the toxins are toxin A or toxin B of C. difficile, or toxin A or toxin B of C. botulinum. In a particular embodiment, the polynucleotide encodes a binding molecule that comprises four separate neutralizing binding components, two of which specifically target and bind to toxin A of C. difficile, and two of which specifically target and bind to toxin B of C. difficile. In another particular embodiment, the polynucleotide encodes a neutralizing binding molecule that comprises four separate binding components, two of which specifically target and bind to toxin A of C. botulinum, and two of which specifically target and bind to toxin B of C. botulinum. In an embodiment of any of the above, the polynucleotide is a deoxyribonucleic acid (DNA). In an embodiment of any of the above, the polynucleotide is a ribonucleic acid (RNA). In a particular embodiment of any of the above, the polynucleotide is mRNA.

In particular aspects, the polynucleotide encodes a binding molecule comprising two binding components, e.g., two different VHHs that target and specifically bind to C. difficile toxin A protein, e.g., at different epitope (or neutralization) sites on the C. difficile toxin A protein, such that the toxin is neutralized by the VHH binding. In a particular embodiment, the polynucleotide encodes a binding molecule comprising two binding components, e.g., two different VHHs, that target and specifically bind to C. difficile toxin B protein, e.g., at different epitope (or neutralization) sites on the C. difficile toxin B protein, such that the toxin is neutralized by the VHH binding. In a particular embodiment, the polynucleotide encodes a binding molecule comprising two binding components, e.g., two different VHHs, that target and specifically bind to C. difficile toxin A proteins, and two binding components, e.g., two different VHHs, that target and bind to C. difficile toxin B proteins, thereby neutralizing both of the C. difficile toxins. In embodiments, the polynucleotide encodes a binding molecule comprising different VHH binding components that target and bind to two C. difficile toxin A proteins, or toxic fragments thereof (e.g., bispecific), to two C. difficile toxin B proteins, or toxic fragments thereof (bispecific), or to two C. difficile toxin A proteins and to two C. difficile toxin B proteins, or toxic fragments thereof (e.g., tetraspecific). In an embodiment, the polynucleotide is DNA. In an embodiment, the polynucleotide is RNA. In a particular embodiment, the polynucleotide is mRNA. In embodiments, the heteromultimeric binding molecule is the protein encoded by the described DNA or RNA polynucleotide.

In other particular aspects, the polynucleotide encodes a binding molecule comprising two binding components, e.g., two different VHHs, that target and specifically bind to Botulinum toxin A protein, e.g., at different epitope (or neutralization) sites on the Botulinum toxin A protein, such that the toxin is neutralized by the VHH binding. In a particular embodiment, the polynucleotide encodes a binding molecule comprising two binding components, e.g., two different VHHs, that target and specifically bind to Botulinum toxin B protein, e.g., at different epitope (or neutralization) sites on the Botulinum toxin B protein, such that the toxin is neutralized by the VHH binding. In a particular embodiment, the polynucleotide encodes a binding molecule comprising two binding components, e.g., two different VHHs, that target and specifically bind to Botulinum toxin A proteins, and two binding components, e.g., two VHHs, that target and bind to Botulinum toxin B proteins, thereby neutralizing both of the Botulinum toxins. In embodiments, the polynucleotide encodes a binding molecule comprising different VHH binding components that target and bind to two Botulinum toxin A proteins, or toxic fragments thereof (e.g., bispecific), to two Botulinum toxin B proteins, or toxic fragments thereof (bispecific), or to two Botulinum toxin A proteins and two Botulinum toxin B proteins, or toxic fragments thereof (e.g., tetraspecific). In an embodiment, the polynucleotide is DNA. In an embodiment, the polynucleotide is RNA. In a particular embodiment, the polynucleotide is mRNA. In embodiments, the heteromultimeric binding molecule is the protein encoded by the described DNA or RNA polynucleotide.

As described herein, the heteromultimeric binding molecules according to the invention comprise a plurality of operably linked, modular components, which include a cleavable secretory domain at one terminus of the binding molecule, typically, the 5′ terminus, which allows for secretion of the binding molecule from cells; one or more epitope tag domains to which an anti-epitope tag antibody specifically binds; and two or more target disease agent binding molecule components that specifically bind to the same or to different target disease agents. In embodiments, the cleavable secretory domain is linked to a first epitope tag domain; the first epitope tag domain is linked to a first target disease agent or toxin binding molecule component; a second target disease agent or toxin protein binding molecule component is linked to a second epitope tag binding domain; the two or more target disease agent or toxin binding molecule components are separated from each other by linkers; each of the two or more target disease agent or toxin binding molecule components specifically binds to non-overlapping portions of the same or of different target disease agent or toxin.

In accordance with the invention, the separation of the two or more target disease agent or toxin binding components by flexible linkers allows for facility of specific binding of the target disease agent or toxin binding components to non-overlapping portions of the same or of different target disease agents or toxins, e.g., multiple target agents or toxins, thereby neutralizing the target disease agents or toxins. In other embodiments, the specific binding of the epitope tag domains by anti-epitope tag antibodies facilitates clearance of the binding molecule from a subject following neutralization of the target disease agent or toxin. In a particular embodiment, the epitope tag comprises the amino acid sequence DELGPRLMGK (SEQ ID NO: 1).

In another of its aspects, the two or more toxin binding components of the heteromultimeric binding molecule specifically bind to the same or to different toxins selected from the group consisting of Botulinum toxin A, Botulinum toxin B, Clostridium toxin, Clostridium difficile toxin A; Clostridium difficile toxin B, or a combination thereof.

In an embodiment, a binding molecule as described herein is a polypeptide encoded by a polynucleotide, such as RNA or DNA. In an embodiment, the cleavable secretory domain of the binding molecule is cleaved from the binding molecule to produce a mature binding molecule. In an embodiment, the two or more target disease agent binding components or toxin binding components of the binding molecule are selected from a single-chain antibody (scFv), more particularly, a recombinant camelid heavy-chain-only antibody (VHH). In a particular embodiment, the two or more target disease agents or toxin binding components of the binding molecule comprise VHH molecules.

In an embodiment, the epitope tag domain of the heteromultimeric binding molecules described herein comprises the amino acid sequence GGGGDELGPRLMGKGGGG (SEQ ID NO: 2) or the amino acid sequence DELGPRLMGK (SEQ ID NO: 1), or a nucleic acid encoding all or a functional portion of these sequences. In an embodiment, the epitope tag domain of the heteromultimeric binding molecules described herein comprises the amino acid sequence Gly Ala Pro Val Pro Tyr Pro Asp Pro Leu Glu Pro Arg (SEQ ID NO: 3) or a nucleic acid encoding all or a functional portion of this sequence. In an embodiment, the one or more linkers included in the heteromultimeric binding molecules described herein comprises a peptide having an amino acid sequence selected from GGGGS (SEQ ID NO: 4), GGGGSGGGGSGGGGS (SEQ ID NO: 5), or a combination thereof, or a nucleotide sequence encoding all or functional portions of these sequences.

In an aspect, the present invention provides a pharmaceutical composition comprising one or more heteromultimeric binding protein molecules as described herein and a pharmaceutically acceptable carrier, excipient, or vehicle.

In an aspect, the heteromultimeric binding molecules as described herein provide products and are used in therapies in which neutralization and/or accelerated clearance of a target toxin protein produced by infectious bacteria, namely, C. difficile or C. botulinum, benefits the health and well-being of a subject. For example, the target toxin protein may be C. difficile toxin A (TcdA), C. difficile toxin B (TcdB) or a combination thereof, or the target toxin protein may C. botulinum toxin A (BtA), C. botulinum toxin B (BtB) or a combination thereof, that intoxicate a subject. In an embodiment, the subject may be at risk of being infected or intoxicated by the target toxin protein. In a particular embodiment, the target toxin protein is C. difficile toxin A. In a particular embodiment, the target toxin protein is C. difficile toxin B (TcdB). In a particular embodiment, the target toxin protein is C. botulinum toxin A. In a particular embodiment, the target toxin protein is C. botulinum toxin B. In a particular embodiment, the target toxin proteins are both C. difficile toxin A and toxin B. In a particular embodiment, the target toxin proteins are both C. botulinum toxin A and toxin B.

In another aspect, the present invention provides a heteromultimeric binding molecule for neutralizing C. difficile toxin A, the binding molecule comprising: a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds; two C. difficile toxin protein binding components which specifically bind to non-overlapping portions of the C. difficile toxin A, or fragments thereof that retain toxin A activity; wherein the first epitope tag domain is operably linked to a first C. difficile toxin A binding component comprising the amino acid sequence SGGGLVQPGGSLRLSCAASGFTLDYSSIGWFRQAPGKEREGVSCISSSGDSTKYADSV KGRFTTSRDNAKNTVYLQMNSLKPDDTAVYYCAAFRATMCGVFPLSPYGKDDWG KGTLVTVSS (SEQ ID NO: 6) and a second C. difficile toxin A binding component comprising the amino acid sequence SGGGLVQPGGSLRLSCAASGFTFSDYVMTWVRQAPGKGPEWIATINTDGSTMRDDS TKGRFTISRDNAKNTLYLQMTSLKPEDTALYYCARGRVISASAIRGAVRGPGTQVTV SS (SEQ ID NO: 7) is operably linked to a second epitope tag binding domain comprising the amino acid sequence DELGPRLMGK as set forth in SEQ ID NO: 1; wherein the C. difficile toxin A binding components are separated from each other by flexible linkers comprising an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof; wherein separation of the C. difficile toxin A binding components by flexible linkers allows for specific binding of the C. difficile toxin A binding components to non-overlapping portions of C. difficile toxin A, thereby neutralizing the C. difficile toxin A protein; and wherein specific binding of the epitope tag domains by an anti-epitope tag antibody facilitates clearance of the binding molecule from a subject following neutralization of the C. difficile toxin A protein.

In another aspect, the present invention provides a heteromultimeric binding molecule for neutralizing C. difficile toxin B, the binding molecule comprising: a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds; two C. difficile toxin protein binding components which specifically bind to non-overlapping portions of the C. difficile toxin B, or fragments thereof that retain toxin B activity; wherein the first epitope tag domain is operably linked to a first C. difficile toxin B binding component comprising the amino acid sequence SGGGLVQPGGSLRLSCEASGFTLDYYGIGWFRQPPGKEREAVSYISASARTILYADSV KGRFTISRDNAKNAVYLQMNSLKREDTAVYYCARRRFSASSVNRWLADDYDVWG RGTQVAVSS (SEQ ID NO: 11) and a second C. difficile toxin B binding component comprising the amino acid sequence SGGGLVQTGGSLRLSCASSGSIAGFETVTWSRQAPGKSLQWVASMTKTNNEIYSDSV KGRFIISRDNAKNTVYLQMNSLKPEDTGVYFCKGPELRGQGIQVTVSS (SEQ ID NO: 12) is operably linked to a second epitope tag binding domain comprising the amino acid sequence DELGPRLMGK as set forth in SEQ ID NO: 1; wherein the C. difficile toxin B binding components are separated from each other by flexible linkers comprising an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof; wherein separation of the C. difficile toxin B binding components by flexible linkers allows for specific binding of the C. difficile toxin B binding components to non-overlapping portions of C. difficile toxin B, thereby neutralizing the C. difficile toxin B protein; and wherein specific binding of the epitope tag domains by an anti-epitope tag antibody facilitates clearance of the binding molecule from a subject following neutralization of the C. difficile toxin B protein.

In another aspect, the present invention provides a heteromultimeric binding molecule for neutralizing C. difficile toxin A and toxin B proteins, the binding molecule comprising: a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds; four C. difficile toxin protein binding components, two of which specifically bind to non-overlapping portions of C. difficile toxin A, or fragments thereof that retain toxin A activity and two of which specifically bind to non-overlapping portions of C. difficile toxin B, or fragments thereof that retain toxin B activity; wherein a first C. difficile toxin A binding component comprises the amino acid sequence SGGGLVQPGGSLRLSCAASGFTLDYSSIGWFRQAPGKEREGVSCISSSGDSTKYADSV KGRFTTSRDNAKNTVYLQMNSLKPDDTAVYYCAAFRATMCGVFPLSPYGKDDWG KGTLVTVSS (SEQ ID NO: 6), and a second C. difficile toxin A binding component comprises the amino acid sequence SGGGLVQPGGSLRLSCAASGFTFSDYVMTWVRQAPGKGPEWIATINTDGSTMRDDS TKGRFTISRDNAKNTLYLQMTSLKPEDTALYYCARGRVISASAIRGAVRGPGTQVTV SS (SEQ ID NO: 7), and wherein a first C. difficile toxin B binding component comprises the amino acid sequence SGGGLVQPGGSLRLSCEASGFTLDYYGIGWFRQPPGKEREAVSYISASARTILYADSV KGRFTISRDNAKNAVYLQMNSLKREDTAVYYCARRRFSASSVNRWLADDYDVWG RGTQVAVSS (SEQ ID NO: 11), and a second C. difficile toxin B binding component comprises the amino acid sequence SGGGLVQTGGSLRLSCASSGSIAGFETVTWSRQAPGKSLQWVASMTKTNNEIYSDSV KGRFIISRDNAKNTVYLQMNSLKPEDTGVYFCKGPELRGQGIQVTVSS (SEQ ID NO: 12); wherein one terminus of the first C. difficile toxin A binding component is flexibly linked to one terminus of the first C. difficile toxin B binding component; a second terminus of the first C. difficile toxin B binding component is flexibly linked to one terminus of the second C. difficile toxin B binding component; a second terminus of the second C. difficile toxin B binding component is flexibly linked to one terminus of the second C. difficile toxin A binding component; and a second terminus of the second C. difficile toxin A binding component is flexibly linked to a second epitope tag binding domain comprising the amino acid sequence DELGPRLMGK as set forth in SEQ ID NO: 1; wherein the linker comprises an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof; wherein separation of the C. difficile toxin binding components by flexible linkers allows for specific binding of the C. difficile toxin binding components to non-overlapping portions of C. difficile toxin A and toxin B, thereby neutralizing the C. difficile toxin A and toxin B proteins; and wherein specific binding of the epitope tag domains by one or more an anti-epitope tag antibodies facilitates clearance of the binding molecule from a subject following neutralization of C. difficile toxin A and toxin B.

In another aspect, the present invention provides a heteromultimeric binding molecule for neutralizing a C. botulinum toxin A protein target disease agent, the binding molecule comprising: a cleavable secretory domain at one terminus of the binding molecule, the domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds; two C. botulinum toxin protein binding components which specifically bind to non-overlapping portions of the C. botulinum toxin A protein, or fragments thereof that retain toxin A activity; wherein the first epitope tag domain is operably linked to a first C. botulinum toxin A binding component comprising the amino acid sequence SGGGLVQVGGSLRLSCVVSGSDISGIAMGWYRQAPGKRREMVADIFSGGSTDYAGS VKGRFTISRDNAKKTSYLQMNNVKPEDTGVYYCRLYGSGDYWGQGTQVTVSS (SEQ ID NO: 13) and a second C. botulinum toxin A binding component comprising the amino acid sequence SGGGLVHPGGSLRLSCAPSASLPSTPFNPFNNMVGWYRQAPGKQREMVASIGLRINY ADSVKGRFTISRDNAKNTVDLQMDSLRPEDSATYYCHIEYTHYWGKGTLVTVSS (SEQ ID NO: 14) is operably linked to a second epitope tag binding domain comprising the amino acid sequence DELGPRLMGK as set forth in SEQ ID NO: 1; wherein the C. botulinum toxin A binding components are separated from each other by flexible linkers comprising an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof; wherein separation of the C. botulinum toxin A binding components by flexible linkers allows for specific binding of the C. botulinum toxin A binding components to non-overlapping portions of C. botulinum toxin A, thereby neutralizing the C. botulinum toxin A protein; and wherein specific binding of the epitope tag domains by an anti-epitope tag antibody facilitates clearance of the binding molecule from a subject following neutralization of the C. botulinum toxin A protein.

In another aspect, the invention provides a heteromultimeric binding molecule comprising two VHH binding regions that specifically bind to and neutralize C. difficile toxin A, wherein the binding molecule has the amino acid sequence set forth in SEQ ID NO: 16. (FIG. 1).

In another aspect, the invention provides a heteromultimeric binding molecule comprising two VHH binding regions that specifically bind to and neutralize C. difficile toxin B, wherein the binding molecule has the amino acid sequence set forth in SEQ ID NO: 17. (FIG. 2).

In another aspect, the invention provides a heteromultimeric binding molecule comprising two VHH binding regions that specifically bind to and neutralize C. difficile toxin A and two VHH binding regions that specifically bind to and neutralize C. difficile toxin B, wherein the binding molecule has the amino acid sequence set forth in SEQ ID NO: 18. (FIG. 3).

In another aspect, the invention provides a heteromultimeric binding molecule comprising two VHH binding regions that specifically bind to and neutralize C. botulinum toxin A, wherein the binding molecule has the amino acid sequence set forth in SEQ ID NO: 19. (FIG. 4).

In embodiments of any of the foregoing aspects, the heteromultimeric binding molecule may optionally include an albumin binding sequence at the 3′ end of the molecule. In an embodiment, such an albumin binding sequence has the amino acid sequence DICLPRWGCLWED as set forth in SEQ ID NO: 15.

In other aspects, a pharmaceutical composition comprising any of the above-described binding molecules and a pharmaceutically acceptable carrier, excipient, or vehicle is provided. In other aspects, a polynucleotide encoding any one of the above-described binding molecules is provided. In an embodiment, the polynucleotide is an RNA molecule (RNA polynucleotide) or a DNA molecule (DNA polynucleotide). In an embodiment, the RNA polynucleotide is a messenger RNA (mRNA) molecule, which is transcribed in a cell. In a particular embodiment, the mRNA polynucleotide has a codon optimized sequence for human expression. In an embodiment, the DNA molecule is a complementary DNA (cDNA) molecule. In an aspect, a pharmaceutical composition is provided, which comprises any one of the foregoing polynucleotides and a pharmaceutically acceptable carrier, excipient, or vehicle.

In another aspect, an expression vector including a polynucleotide as described above is provided. In an embodiment, the expression vector includes an operably linked promoter sequence, signal sequence and stop codon sequence. In an embodiment, the expression vector is a bacterial, eukaryotic or viral vector.

In another aspect, a prokaryotic or eukaryotic host cell comprising the above-described expression vector is provided.

In another aspect, a lipid nanoparticle is provided which comprises an mRNA polynucleotide. In an embodiment, the mRNA polynucleotide has a codon optimized sequence for human expression. In an aspect, a pharmaceutical composition is provided which comprises the above-described lipid nanoparticle and a pharmaceutically acceptable carrier, excipient, or vehicle.

In another aspect, a method of neutralizing a target disease agent or a toxin is provided, in which the method comprises contacting a target disease agent or toxin with a binding molecule, a polynucleotide, or a pharmaceutical composition as described above in an amount effective for the binding molecule to bind to and neutralize the target disease agent or the toxin. In an embodiment of the method, the target disease agent or toxin is neutralized in vitro or in vivo. In an embodiment of the method, the binding molecule contacts the target disease agent or the toxin in a cell.

In another aspect, a method of treating or preventing intoxication of a subject by a toxin is provided, in which the method comprises administering to a subject in need thereof a binding molecule, polynucleotide, or pharmaceutical composition as described above in an effective amount for the binding molecule to neutralize the toxin in the subject after intoxication or prior to the subject's having symptoms of intoxication.

In another aspect, a method of neutralizing a target disease agent or a toxin is provided, in which the method comprises contacting a target disease agent or the toxin with the above-described lipid nanoparticle comprising mRNA encoding a heteromultimeric VNA binding molecule as described herein in an amount effective for expression of the binding molecule which binds to and neutralizes the target disease agent or the toxin.

In another aspect, a method of treating or preventing intoxication of a subject by a toxin is provided, in which the method comprises administering to a subject in need thereof the above-described lipid nanoparticle comprising mRNA encoding a heteromultimeric VNA binding molecule as described herein in an amount effective for expression of the binding molecule which neutralizes the toxin in the subject after intoxication or prior to the subject's having symptoms of intoxication.

In embodiments of any of the foregoing methods, the binding molecule, polynucleotide, or pharmaceutical composition is administered enterally or parenterally. In embodiments of the foregoing methods, the target disease agent or toxin is selected from C. difficile toxin A, C. difficile toxin B, C. difficile toxin A and C. difficile toxin B, Botulinum toxin A, Botulinum toxin B, or Botulinum toxin A and Botulinum toxin B.

In another aspect, the invention provides the above-described heteromultimeric binding molecules which are recombinantly produced.

In other aspects, the invention provides kits comprising a heteromultimeric binding molecule, a polynucleotide, or a pharmaceutical composition as described supra and infra, for treating or monitoring a subject exposed to or at risk of exposure to infection by C. difficile or C. botulinum and disease resulting therefrom. In an aspect, a kit is provided which comprises a lipid nanoparticle as described supra and infra, for treating or monitoring a subject exposed to or at risk or exposure to infection by C. difficile or C. botulinum and/or to disease resulting therefrom.

Other features and advantages of the invention will be apparent from the detailed description and from the claims infra.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, reduce, diminish, suppress, attenuate, arrest, or stabilize the development or progression of a disease or pathology.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant any immunoglobulin polypeptide, or fragment thereof, having immunogen binding ability.

By “binding to” a molecule is meant having a physicochemical affinity for that molecule. Binding may be measured by any of the methods practiced in the art, e.g., using an antibody binding assay or an in vitro translation binding assay.

“Detect” refers to identifying or determining the presence, absence or amount of an analyte to be detected.

By “detectable label” is meant a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include, without limitation, C. difficile infection (CDI), C. difficile-associated diarrhea (CDAD), pseudomembranous colitis (PMC), bowel inflammation, enterocytic detachment, alteration, disruption, or elimination of natural intestinal microflora, and/or paralytic ileus caused by C. difficile infection, or to botulinum disease or wounds resulting from Botulinum infection, or one or more symptoms of these diseases. The production of toxins by the pathogenic and infectious C. difficile and C. botulinum microorganisms results in intoxication of the subject (or patient), which is an abnormal state that is a poisoning of the subject (and the subject's cells, tissues and organs) by the presence and activity of the produced toxins.

By “effective amount” is meant the amount of a required to ameliorate, or optimally eliminate, the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, 90%, or 95% of the entire length of the reference nucleic acid molecule or polypeptide, including percent values between those enumerated. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the terms “polynucleotide,” “DNA molecule” or “nucleic acid molecule” include both sense and anti-sense strands, cDNA, genomic DNA, recombinant DNA, RNA, mRNA, and wholly or partially synthesized nucleic acid molecules. A nucleotide “variant” is a sequence that differs from the recited nucleotide sequence in having one or more nucleotide deletions, substitutions or additions. Such modifications are readily introduced using standard mutagenesis techniques, such as oligonucleotide-directed site-specific mutagenesis as described, for example, in Adelman et al., 1983, DNA 2:183. Nucleotide variants are naturally-occurring allelic variants, or non-naturally occurring variants. Variant nucleotide sequences in various embodiments exhibit at least about 70%, 75%, 80%, 85%, 90%, 95%, 98%, 99%, or 100% sequence homology or sequence identity to the recited sequence. Such variant nucleotide sequences hybridize to the recited nucleotide sequence under stringent hybridization conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

By “isolated polynucleotide” is meant a nucleic acid (e.g., DNA, cDNA, RNA, mRNA) that is free of the genes, which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, e.g., mRNA, as well as a recombinant DNA that is part of a hybrid gene encoding additional polypeptide sequence.

The terms “protein”, “peptide” and “polypeptide” are used herein to describe any chain of amino acid residues, regardless of length or post-translational modification (for example, glycosylation or phosphorylation). Thus, these terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length proteins, which may be, but need not be, naturally occurring, as well as recombinantly or synthetically produced polypeptides that correspond to a full-length protein, or to particular domains or portions of a protein, which may be, but need not be, naturally occurring. The term also encompasses mature proteins which have an added amino-terminal methionine to facilitate expression in prokaryotic cells. The binding molecules of the invention are encoded by polynucleotides and can be chemically synthesized or synthesized by recombinant DNA methods.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

By “pathogen” is meant any harmful microorganism, bacterium, virus, fungus, or protozoan capable of interfering with the normal function of a cell. Pathogens as referred to herein produce toxins, e.g., protein toxins, that intoxicate the cells and tissues of a host or recipient organism and cause disease and pathology, often severe, unless they are neutralized and eliminated from the organism to the extent possible, such as by action of the VNA binding molecules described herein. As described herein, bacterial pathogens include, but are not limited to, Clostridium difficile and Clostridium botulinum, which produce toxin proteins that intoxicate a subject after infection. Toxin A and toxin B proteins produced by C. difficile translocate to the cytosol of target cells (mainly of the intestinal epithelium, but also in other cells) and inactivate cellular GTP-binding proteins, e.g., Rho, Rac and Cdc42, by monoglucosylation, which causes actin condensation, cell rounding and death. (reviewed by D. Voth and D. Ballard, 2005, Clin. Microbiol. Rev., Vol. 18 (2):247-263). Serotype A and B toxins (endotoxins) produced by C. botulinum are neurotoxins that interfere with neural transmission by blocking or preventing the release of acetylcholine in muscle cells, thereby preventing muscle cell contraction and causing muscle paralysis. (P. K. Nigam and A. Nigam, 2010, Indian J. Dermatol., 55(1):8-14).

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative or lowering alteration of at least 5%, 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound, molecule, or antibody that recognizes and binds a protein, peptide, or polypeptide (e.g., an amino acid sequence of the protein, peptide, or polypeptide), but which does not substantially recognize and bind other molecules in a sample, for example, a biological sample, which may contain the protein, peptide, or polypeptide that is specifically bound.

“Nucleic acid” (also called polynucleotide herein) refers to deoxyribonucleotides or ribonucleotides and polymers thereof in either single- or double-stranded form. The term encompasses nucleic acids (polynucleotides) containing known nucleotide analogs or modified backbone residues or linkages, which are synthetic, naturally occurring, and non-naturally occurring, which have similar binding properties as a reference nucleic acid, and which are metabolized in a manner similar to the reference nucleic acid. Examples of such analogs include, without limitation, phosphorothioates, phosphoramidates, methyl phosphonates, chiral methyl phosphonates, 2-O-methyl ribonucleotides, and peptide-nucleic acids (PNAs). Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., 1991, Nucleic Acid Res, 19:081; Ohtsuka et al., 1985, J Biol. Chem., 260:2600-2608; Rossolini et al., 1994, Mol. Cell Probes, 8:91-98). The term nucleic acid can be used interchangeably with gene, cDNA, mRNA, oligonucleotide, and polynucleotide.

Nucleic acid molecules or polynucleotides useful in the invention include any nucleic acid molecule or polynucleotide that encodes a polypeptide, e.g., a heteromultimeric binding molecule, of the invention or a component or portion thereof. Nucleic acid molecules useful in the methods of the invention include any polynucleotide or nucleic acid molecule that encodes a polypeptide e.g., heteromultimeric binding molecule, of the invention or a component or portion thereof that has substantial identity to the binding molecule. Such nucleic acid molecules need not be 100% identical with the nucleic acid sequence of the binding molecule, but will typically exhibit substantial identity. Polynucleotides having “substantial identity” to a binding molecule sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, 1987, Methods Enzymol. 152:399; Kimmel, A. R., 1987, Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and even more preferably at least 95% sequence identity.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as, without limitation, a human, a non-human primate, or a bovine, equine, canine, ovine, or feline mammal. Other mammals include rabbits, goats, llamas, mice, rats, guinea pigs, camels and gerbils. In particular, a “subject” as used herein refers to a human subject, such as a human patient. In some cases, the terms subject and patient are used interchangeably herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

A VHH binding molecule as referred to herein is, in general, a single domain immunoglobulin molecule (antibody) isolated from camelid animals. A VHH (or VHH antibody) corresponds to the heavy chain of a camelid antibody having a single variable domain (or single variable region), e.g., a camelid-derived single variable H (V_{H}) domain antibody. A VHH has a molecular weight (MW) of about 15 kDa. VHH technology is based on fully functional antibodies from camelids that lack light chains. These heavy-chain antibody molecules contain a single variable domain (VHH) and two constant domains (CH₂ and CH₃). A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety. VHHs, called NANOBODIES™, may be obtained commercially (Ablynx Inc., Ghent, Belgium).

VHHs are efficiently expressed in E. coli, coupled to detection markers, such as a fluorescent marker, or conjugated with enzymes. The small size of VHHs permits their binding to epitopes, e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage.

A VHH-based binding molecule or polypeptide that specifically binds to and neutralizes the activity of a target agent, such as a bacterial toxin, is referred to as a “VHH-based neutralizing agent (VNA)” a “VNA polypeptide molecule” or a “VNA binding molecule” herein.

As used herein, the terms “treat,” treating,” “treatment,” and the like refer to reducing, diminishing, abating, alleviating, improving, or ameliorating a disorder and/or symptoms associated therewith. It will be appreciated that, although not precluded, treating a disorder or condition does not require that the disorder, condition or symptoms associated therewith be completely eliminated.

As used herein, a “multimeric binding molecule” refers in general to a multicomponent protein or polypeptide containing two or more, same or different, VHH binding molecules, which are coupled or linked, e.g., via spacer sequences, to other components of the molecule. Multimeric binding molecules may be homomultimeric, in that the binding molecule contains more than one, e.g., two, different VNA binding molecule components that bind to the same target agent, e.g., toxin A of C. difficile, or to toxin B of C. difficile, or to Botulinum toxin A. The different VNA binding molecule components of a homomultimeric binding molecule may bind to different regions, portions, or epitopes (e.g., non-overlapping epitopes) of the same target agent. Alternatively, the multimeric binding molecules may be heteromultimeric, in that the binding molecule contains more than one, e.g., two or four, different VHH binding molecule components, each of which specifically binds to a different target agent (e.g., a toxin protein) or to different regions, portions, or epitopes (non-overlapping epitopes) of the same target agent, such that the heteromultimeric binding molecule comprises several different VHH binding molecule components, for example, two or four different VHH binding molecule components. In some embodiments, the heteromultimeric binding molecule contains two different VHH binding molecule components, each of which specifically binds to toxin A of C. difficile, e.g., at non-overlapping epitopes, and also contains two different VHH binding molecule components, each of which specifically binds to toxin B of C. difficile. A VNA binding molecule can refer to a heteromultimeric binding molecule that comprises two or more different VHH binding molecule components.

As used herein, the terms “prevent,” “preventing,” “prevention,” “prophylactic treatment,” “protection” and the like refer to reducing the probability of developing a disorder or condition in a subject, who does not have, but who is at risk of, is susceptible to, or disposed to (e.g., genetically disposed to), developing a disease, disorder, pathology, or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Any compositions or methods provided herein can be combined with one or more of any of the other compositions and methods provided herein.

### BRIEF DESCRIPTION OF THE DRAWINGS

FIG. 1 presents the amino acid sequence of a bispecific VHH-based neutralizing agent (VNA) polypeptide molecule that specifically binds to two Clostridium difficile toxin A (TcdA) proteins and neutralizes TcdA activity (SEQ ID NO: 16). Such a bispecific VHH-based neutralizing agent (VNA) polypeptide molecule may also be referred to as a VHH heterodimer, or more generally, a heteromultimeric binding molecule. In FIG. 1, two epitope tags (O-tag epitopes) are present in the molecule, as shown in bold italics. The minimal 0-tag epitope amino acid sequence is DELGPRLMGK (SEQ ID NO: 1). The two VHH C. difficile toxin A binding regions of the VNA polypeptide molecule are identified in FIG. 1 by double underlining. An optional albumin binding peptide (DICLPRWGCLWED, SEQ ID NO: 15) is shown at the 3′ end of the molecule by broken line underlining.

FIG. 2 presents the amino acid sequence of a bispecific VHH-based neutralizing agent (VNA) polypeptide molecule that specifically binds to two Clostridium difficile toxin B (TcdB) proteins and neutralizes TcdB activity (SEQ ID NO: 17). Such a bispecific VHH-based neutralizing agent (VNA) polypeptide molecule may also be referred to as a VHH heterodimer, or more generally, a heteromultimeric binding molecule. In FIG. 2, two epitope tags (O-tag epitopes) are present in the molecule, as shown in bold italics. The minimal 0-tag epitope amino acid sequence is DELGPRLMGK (SEQ ID NO: 1). The two VHH C. difficile toxin A binding regions of the VNA polypeptide molecule are identified in FIG. 1 by double underlining. An optional albumin binding peptide (DICLPRWGCLWED, SEQ ID NO: 15) is shown at the 3′ end of the molecule by broken line underlining.

FIG. 3 presents the amino acid sequence of a tetraspecific VHH-based neutralizing agent (VNA) polypeptide molecule that specifically binds to two Clostridium difficile toxin A (TcdA) proteins and neutralizes TcdA activity and also binds to two Clostridium difficile toxin B (TcdB) proteins and neutralizes TcdB activity (SEQ ID NO: 18). Such a tetraspecific VHH-based neutralizing agent (VNA) polypeptide molecule may also be referred to as a VHH heteromultimeric binding molecule. In FIG. 3, two epitope tags (O-tag epitopes) are present in the molecule, as shown in bold italics. The minimal O-tag epitope amino acid sequence is DELGPRLMGK (SEQ ID NO: 1). The two VHH C. difficile toxin A binding regions of the VNA polypeptide molecule are identified in FIG. 3 by double underlining and the two VHH C. difficile toxin B binding regions of the VNA polypeptide molecule are identified in FIG. 3 by dotted underlining. An optional albumin binding peptide (DICLPRWGCLWED, SEQ ID NO: 15) is shown at the 3′ end of the molecule by broken line underlining.

FIG. 4 presents the amino acid sequence of a bispecific VHH-based neutralizing agent (VNA) polypeptide molecule that specifically binds to two Botulinum toxin A (BtA) proteins and neutralizes BtA activity (SEQ ID NO: 19). Such a bispecific VHH-based neutralizing agent (VNA) polypeptide molecule may also be referred to as a heterodimeric binding molecule, or more generally, a heteromultimeric binding molecule. In FIG. 4, two epitope tag amino acid sequences (O-tag epitopes) are present in the molecule, as shown in bold italics. The minimal epitope tag amino acid sequence is DELGPRLMGK (SEQ ID NO: 1). The two VHH Botulinum toxin A binding regions of the VNA polypeptide molecule are identified in FIG. 4 by double underlining. An optional albumin binding peptide (DICLPRWGCLWED, SEQ ID NO: 15) is shown at the 3′ end of the molecule by broken line underlining.

FIGS. 5A and 5B present the heavy and light chains of an anti-epitope tag (O-tag) monoclonal antibody (mAb), a mouse effector IgG1 mAb (efAb) that binds to the O-tag epitope of a multimeric VNA binding protein as described herein. FIG. 5A presents the amino acid sequence of the heavy chain of the murine IgG1 mAb (anti-O-tag mAb heavy chain molecule), (SEQ ID NO: 20), that binds to the epitope tag (O-tag) amino acid sequence DELGPRLMGK (SEQ ID NO: 1), i.e., an anti-O-tag mAb heavy chain molecule, which is involved in clearance from the body of the multimeric binding molecule that has bound to its toxin target molecules. FIG. 5B presents the amino acid sequence of the light chain of the murine IgG1 mAb (anti-O-tag mAb light chain molecule), (SEQ ID NO: 21) that binds to the epitope tag (O-tag) amino acid sequence DELGPRLMGK (SEQ ID NO: 1).

FIGS. 6A-6D present graphs showing the results of either ELISAs or toxin neutralization assays employing the VNAs as described herein. FIG. 6A illustrates the results of an ELISA in which RNA encoding the C. difficile toxin A (TcdA) binding molecule of FIG. 1 (VNA1-TcdA) and RNA encoding the C. difficile toxin A (TcdA) and C. difficile toxin B (TcdB) binding molecule of FIG. 3 (VNA2-Tcd) were produced as RNA nanoparticles, which were used to transfect baby hamster kidney (BHK) cells. Supernatants from the transfected cells were collected 48 hours post-transfection and dilutions of the supernatants were assayed by ELISA as shown and as described in Example 1 herein. As observed in FIG. 6A, both VNA1-TcdA, which targets only TcdA, and VNA2-Tcd, which targets TcdA and TcdB, bind to TcdA in the ELISA even at dilutions greater than 1:500 relative to conditioned medium control (supernatant from cells transfected no VNA or with an irrelevant VNA molecule). FIG. 6C illustrates the results of an ELISA in which RNA encoding the C. difficile toxin B (TcdB) binding molecule of FIG. 2 (VNA1-TcdB) and RNA encoding the C. difficile toxin B (TcdA) and C. difficile toxin B (TcdB) binding molecule of FIG. 3 (VNA2-Tcd) were produced as RNA nanoparticles, which were used to transfect baby hamster kidney (BHK) cells as described for FIG. 6A. As observed in FIG. 6C, both VNA1-TcdB, which targets only TcdB, and VNA2-Tcd, which targets TcdA and TcdB, bind to TcdB in the ELISA even at dilutions greater than 1:500 relative to the conditioned medium control. FIGS. 6B and 6D show the results of standard toxin neutralization assays in which the transfected BHK cell supernatants as described above were serially diluted and contacted with Vero cells that were treated with either TcdA or TcdB at known toxic levels. Toxicity of cells was assessed by quantifying the percent rounded cells. FIG. 6B assessed the neutralization activity of VNA1-TcdA and VNA2-Tcd against TcdA intoxication of Vero cells. FIG. 6D assessed the neutralization activity of VNA1-TcdB and VNA2-Tcd against TcdB intoxication of Vero cells. Conditioned medium from the VNA transfected samples fully protected cells against the appropriate toxin (low % cell rounding) even when diluted >100 fold and partially protected cells from the toxin at a dilution of 1:1000 or higher. The control was medium alone, which did not protect cells from intoxication, such that all cells affected by the toxin were 100% rounded.

### DETAILED DESCRIPTION OF THE EMBODIMENTS

Provided herein are binding protein molecules, compositions and methods that are useful for treating or preventing diseases and pathologies associated with infection by pathogenic microorganisms (bacteria), in particular, Clostridium microorganisms or Botulinum microorganisms. The binding molecules, compositions and method are also useful for treating or preventing diseases, illness and pathologies caused by the toxins produced by these microorganisms following infection. In an embodiment, the microorganisms are Clostridium difficile (C. difficile) bacteria, which cause C. difficile infection (CDI) and which produce and secrete toxins that disrupt or corrupt the normal gut microflora and microbiome and cause gastrointestinal diseases, including diarrhea, toxemia and toxic megacolon in infected subjects (patients). In many instances, infection by C. difficile is nosocomial (hospital-acquired) and the resulting pathologies associated with the C. difficile toxins can be life-threatening. In embodiments, the toxins produced by C. difficile include toxin A (TcdA), toxin B (TcdB) and toxin C (TcdC). In an embodiment, the primary virulence factors of C. difficile infection are TcdA and TcdB. In another embodiment, the microorganisms are Botulinum bacteria and species thereof, which produce botulinum toxins of serotypes A-G. In an embodiment, the toxin is Botulinum toxin serotype A (Botulinum toxin A, (BtA)).

Provided and described herein are heteromultimeric binding protein molecules that are designed to contain at least two, individual VHH binding protein components that bind to and neutralize at least two target disease agents, namely, a toxin protein (or peptide) produced by C. difficile or C. botulinum. The VHH binding proteins comprise single chain immunoglobulin (antibody) molecules generated by the Camelidae species, in particular, of the IgG class, which includes, without limitation, the IgG1, IgG2 (IgG2a, IgG2b), IgG3 and IgG4 isotypes or subtypes. The recombinantly produced, heteromultimeric binding protein molecules comprise VHH binding protein components and other components that are linked, e.g., covalently linked to produce bispecific heterodimers or tetraspecific heteromultimers, more generally called “heteromultimeric binding proteins” or “heteromultimeric binding protein molecules” herein. The VHH binding protein components are separated by flexible spacer peptides within the heteromultimeric binding molecules.

The heteromultimeric binding protein molecules which comprise at least two VHH binding protein components are also referred to herein as “VHH-based neutralizing agents,” “VNAs,” “VNA binding molecules,” or “VNA molecules,” which specifically bind to and neutralize the activity of two or more target disease agents, such as toxin proteins produced by infectious, pathogenic Clostridium difficile or Clostridium botulinum bacteria. In general, the at least two VHH binding protein components bind and neutralize a target disease agent, such as a bacterial toxin protein, but are different from each other (i.e., they have non-identical sequences). Thus, in one embodiment, a VNA molecule may contain two VHH binding proteins, each of which binds to and neutralizes the activity of two C. difficile TcdA toxin proteins. In another embodiment, a VNA molecule may contain two VHH binding proteins, each of which binds to and neutralizes the activity of two C. difficile TcdB toxin proteins. In another embodiment, a VNA molecule may contain two VHH binding proteins, one of which binds to and neutralizes the activity of a C. difficile TcdA toxin protein and the other of which binds to and neutralizes the activity of a C. difficile TcdB toxin protein. In another embodiment, a VNA molecule may contain four VHH binding proteins, two of which bind to and neutralize the activity of two C. difficile TcdA toxin proteins and two of which bind to and neutralize the activity of two C. difficile TcdB toxin proteins. In another embodiment, a VNA molecule may contain two different VHH binding proteins, each of which binds to and neutralizes the activity of two C. botulinum toxin A proteins. In particular embodiments related to the described VNA binding molecules, the two or more VHH binding protein components that bind to and neutralize a given toxin protein, e.g., TcdA, TcdB, or BtA, are non-identical to each other.

By way of example, a VNA binding molecule according to the invention may be bispecific, that is, the VNA is designed to contain two VHH component monomers, each of which binds to and neutralizes the activity of two toxin proteins. It will be understood that the terms “VHH binding protein (molecule)” and “VHH component monomer” are used interchangeably herein. In an embodiment, the VHH component monomers are different (non-identical in amino acid sequence) and bind to different and/or non-overlapping epitopes on the toxin proteins. A heteromultimeric binding molecule containing two, toxin binding VHH component monomers may be termed a bispecific VNA polypeptide molecule. In an embodiment, the two different VHH component monomers of the bispecific binding molecule bind to and neutralize the activity of two TcdA molecules. In an embodiment, the two different VHH component monomers bind to and neutralize the activity of two TcdB molecules. In an embodiment, the two different VHH component monomers bind to and neutralize the activity of a TcdA molecule and a TcdB molecule. In an embodiment, the two different VHH component monomers bind to and neutralize the activity of two Botulinum toxin A molecules. In an embodiment, the two different VHH component monomers bind to and neutralize the activity of two Botulinum toxin B molecules. In an embodiment, the two different VHH component monomers bind to and neutralize the activity of a Botulinum toxin A molecule and a Botulinum toxin B molecule.

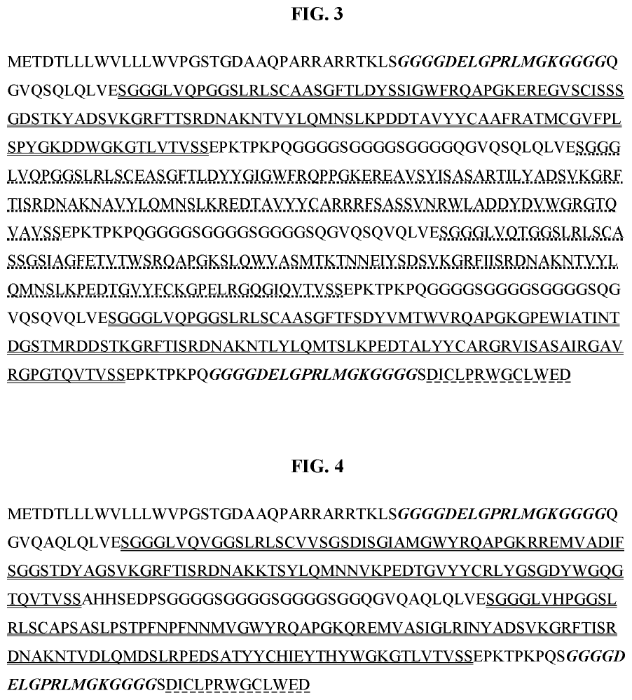

By way of further example, a VNA binding molecule according to the invention may be tetraspecific, that is, the VNA binding molecule is designed to contain four different VHH component monomers, each of which binds to and neutralizes the activity of a toxin protein. A VNA binding molecule containing four different VHH component monomers may be multispecific, such as tetraspecific. In an embodiment, the tetraspecific VNA binding molecule comprises two different VHH component monomers, each of which binds to (and neutralizes) a toxin protein of one type and two different VHH component monomers, each of which binds to (and neutralizes) a toxin protein of another type (e.g., FIG. 3, which shows the amino acid sequence of a multimeric VNA binding molecule comprising two different VHH binding proteins that bind C. difficile TcdA toxin protein and two different VHH binding proteins that bind C. difficile TcdB toxin protein). In another embodiment, the tetraspecific VNA binding molecule comprises four different VHH component monomers, each of which binds to four of the same toxin protein, preferably at different and/or non-overlapping epitopes of the toxin proteins, e.g., C. difficile or Botulinum toxin proteins. In another embodiment, the tetraspecific VNA binding molecule contains two different VHH component monomers that bind to and neutralize the activity of two TcdA toxin proteins and two different VHH component monomers that bind to and neutralize the activity of two TcdB toxin proteins. In another embodiment, the tetraspecific VNA binding molecule contains two different VHH component monomers that bind to and neutralize the activity of two Botulinum toxin A proteins and two different VHH component monomers that bind to and neutralize the activity of two Botulinum toxin B proteins.

### Polynucleotides Encoding VNA Binding Molecules

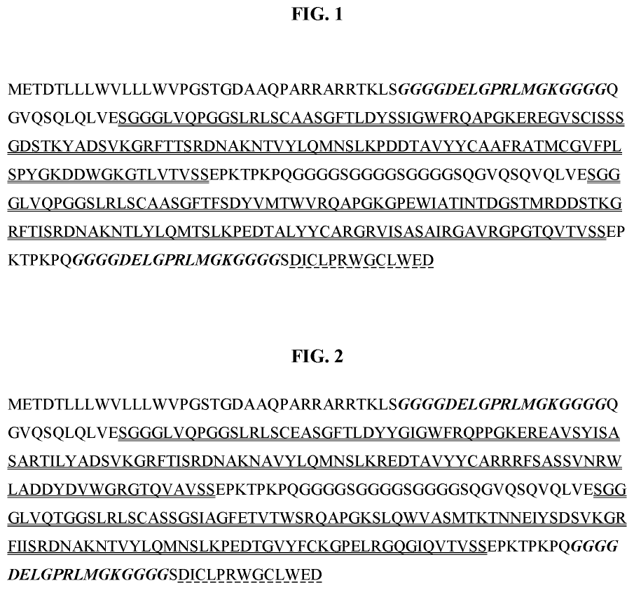

In the VNA binding molecules, the VHH binding components are coupled or linked (e.g., covalently linked) to other sequences, e.g., a leader amino acid sequence, one or more spacer (flexible spacer) amino acid sequences, one or more epitope tag amino acid sequences, to produce a heteromultimeric binding molecule as described herein. (See, e.g., FIGS. 1-5A and 5B). In an embodiment, polynucleotide molecule, such as a recombinant or isolated polynucleotide molecule, encodes a VNA binding protein molecule as described herein. In an embodiment, the polynucleotide encodes a fragment or portion of the VNA binding protein molecule, in particular, a fragment or portion that maintains toxin protein binding and neutralizing function. In an embodiment the polynucleotide encodes a VNA binding protein molecule, or a functional binding portion thereof that includes an epitope tag. In an embodiment, the one or more VHH binding protein component is produced as part of the heteromultimeric VNA binding protein molecule. In an embodiment, antibody fragments, microproteins, darpins, anticalins, adnectins, peptide mimetic molecules, aptamers, synthetic molecules, etc. can be linked to the VNA binding protein molecule. Any combination of VHH binding protein components can be linked to each other and to other sequences to produce a multimeric VNA binding protein molecule. In an embodiment, the VHH binding protein components of the multimeric binding protein molecule are linked covalently.

In another embodiment, a VHH binding protein monomer, or a binding region of the multimeric binding protein molecule, can be modified, for example, by attachment (e.g., directly or indirectly via a linker or spacer) to another VHH binding protein monomer. In some embodiments, a VHH binding protein monomer is attached or genetically (recombinantly) fused to another VHH binding protein monomer. Accordingly, the polynucleotide (DNA) that encodes one VHH binding protein monomer is joined (in reading frame) with the DNA encoding a second VHH binding protein monomer, and so on. In certain embodiments, additional amino acids are encoded within the polynucleotide between the VHH binding protein monomers so as to produce an unstructured region (e.g., a flexible spacer) that separates the VHH binding protein monomers, e.g., to better promote independent folding of each VHH binding protein monomer into its active conformation or shape. Commercially available techniques for fusing proteins (or their encoding polynucleotides) may be employed to recombinantly join or couple the VHH binding protein monomers into the multimeric VNA binding protein molecules containing two or more of the same or different VHH binding proteins as described herein.

Polynucleotide sequences encoding the heteromultimeric toxin-binding VHH molecules as described herein can be recombinantly expressed and the resulting encoded VHH protein molecules can be produced at high levels and isolated and/or purified. In an embodiment, the recombinant VHH protein molecules are produced in soluble form. In an embodiment, the recombinantly produced anti-C. difficile toxin VHH molecule is specific for binding TcdA, TcdB, or a combination of TcdA and TcdB. In an embodiment, a single recombinantly produced anti-C. difficile toxin VHH molecule is bispecific (i.e., the VHH molecule binds to two C. difficile toxin proteins, e.g., two TcdA proteins or two TcdB proteins). In an embodiment, a single recombinantly produced anti-C. difficile toxin VHH molecule is tetraspecific (i.e., the VHH molecule binds to two C. difficile toxin TcdA proteins and to two TcdB proteins). In an embodiment, a single recombinantly produced anti-Botulinum toxin VHH molecule is bispecific (i.e., the VHH molecule binds to two Botulinum toxin proteins, e.g., two BtA proteins). In an embodiment, the bispecific and tetraspecific VNA molecules are contained in pharmaceutically acceptable compositions for treating CDI. In an embodiment, a bispecific VNA molecule is contained in a pharmaceutically acceptable composition for treating toxicity caused by infection Botulinum bacteria, which produce toxins.

The compositions and methods described herein in various embodiments include an isolated polynucleotide sequence or an isolated polynucleotide molecule that encodes a VNA binding protein molecule. Accordingly, the isolated polynucleotide sequence or isolated polynucleotide molecule comprises or consists of a polynucleotide sequence that encodes a polypeptide molecule having an amino acid sequence of SEQ ID NOs: 16-19, or a functional portion thereof, as described herein. (See, FIGS. 1-5A and 5B). In an embodiment, a composition comprises a combination of the isolated polynucleotide sequences or isolated polynucleotide molecules as described herein.

Also encompassed by the present invention are polynucleotide sequences, DNA or RNA, which are substantially complementary to the DNA sequences encoding the polypeptides described herein, and which specifically hybridize with these DNA sequences under conditions of stringency known to those of skill in the art. As referred to herein, substantially complementary means that the nucleotide sequence of the polynucleotide need not reflect the exact sequence of the original encoding sequences, but must be sufficiently similar in sequence to permit hybridization with a nucleic acid sequence under high stringency conditions. For example, non-complementary bases can be interspersed in a nucleotide sequence, or the sequences can be longer or shorter than the polynucleotide sequence, provided that the sequence has a sufficient number of bases complementary to the sequence to allow hybridization thereto. Conditions for stringency are described, e.g., in Ausubel, F. M., et al., Current Protocols in Molecular Biology, (Current Protocol, 1994), and Brown, et al., Nature, 366:575 (1993); and further defined in conjunction with certain assays.

Vectors, plasmids or viruses containing one or more of the polynucleotide molecules encoding the amino acid sequence of SEQ ID NOS: 1-4 are also provided. Suitable vectors for use in eukaryotic and prokaryotic cells are known in the art and are commercially available or readily prepared by the skilled practitioner in the art. Additional vectors can also be found, for example, in Ausubel, F. M., et al., Ibid. and in Sambrook et al., “Molecular Cloning: A Laboratory Manual,” 2nd ED. (1989), and other editions.

Any of a variety of expression vectors (prokaryotic or eukaryotic) known to and used by those of ordinary skill in the art may be employed to express recombinant polypeptides described herein. Expression can be achieved in any appropriate host cell that has been transformed or transfected with an expression vector containing a DNA molecule that encodes a recombinant polypeptide. Suitable host cells include prokaryotes, yeast and higher eukaryotic cells. By way of example, the host cells employed include, without limitation, E. coli, yeast, insect cells, or a mammalian cell line such as COS or CHO. The DNA sequences expressed in this manner can encode any of the polypeptides described herein, including variants thereof.

Uses of plasmids, vectors or viruses containing polynucleotides encoding the VNA protein molecules as described herein includes generation of mRNA or protein in vitro or in vivo. In related embodiments, host cells transformed with the plasmids, vectors, or viruses are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example, yeast (such as Pichia pastoris or Saccharomyces cerevisiae), bacteria (such as E. coli, or Bacillus subtilis), animal cells or tissue (CHO or COS cells), insect Sf9 cells (such as baculoviruses infected SF9 cells), or mammalian cells (somatic or embryonic cells, Human Embryonic Kidney (HEK) cells, Chinese hamster ovary (CHO) cells, HeLa cells, human 293 cells and monkey COS-7 cells). Suitable host cells also include a mammalian cell, a bacterial cell, a yeast cell, an insect cell, and a plant cell.

A VNA protein-encoding polynucleotide molecule can be incorporated or inserted into the host cell by known methods. Examples of suitable methods for transfecting or transforming host cells include, without limitation, calcium phosphate precipitation, electroporation, microinjection, infection, lipofection and direct uptake. “Transformation” or “transfection” as appreciated by the skilled practitioner refers to the acquisition of new or altered genetic features by the incorporation of additional nucleic acids, e.g., DNA, into cellular DNA. “Expression” of the genetic information of a host cell is a term of art which refers to the directed transcription of DNA to generate RNA that is, in turn, translated into a polypeptide. Procedures for preparing recombinant host cells and incorporating nucleic acids are described in more detail in Sambrook et al., “Molecular Cloning: A Laboratory Manual,” Second Edition (1989) and Ausubel, et al. “Current Protocols in Molecular Biology,” (1992), and later editions, for example.

A transfected or transformed host cell is maintained under suitable conditions for expression and recovery of the polypeptides described herein. In certain embodiments, the cells are maintained in a suitable buffer and/or growth medium or nutrient source for growth of the cells and expression (and secretion) of the gene product(s) into the growth medium. The type of growth medium is not critical to the invention and is generally known to those skilled in the art, such as, for example, growth medium and nutrient sources that include sources of carbon, nitrogen and sulfur. Examples include Luria-Bertani (LB) broth, Superbroth, Dulbecco's Modified Eagles Media (DMEM), RPMI-1640, M199 and Grace's insect media. The growth medium can contain a buffering agent, as commonly used in the art. The pH of the buffered growth medium may be selected and is generally a pH that is tolerated by, or optimal for, growth of the host cell, which is maintained under a suitable temperature and atmosphere.

In another aspect, an RNA polynucleotide, in particular, mRNA, encodes the VNA molecules described herein. mRNA encoding the VNAs described herein may contain a 5′ cap structure, a 5′ UTR, an open reading frame, a 3′ UTR and poly-A sequence followed by a C30 stretch and a histone stem loop sequence (Thess, A. et al., 2015, Mol Ther, 23(9):1456-1464; Thran, M. et al., 2017, EMBO Molecular Medicine, DOI: 10.15252/emmm.201707678). Sequences may be codon-optimized for human use. In an embodiment, the mRNA sequences do not include chemically modified bases. mRNAs encoding the VNAs as described herein may be capped enzymatically or further polyadenylated for in vivo studies/use.

Expression of proteins, which normally have a shortened serum half-life, by encoding mRNA, particularly sequence optimized, unmodified mRNA, advantageously prolongs the bioavailability of these proteins for in vivo activity. (see, e.g., K. Kariko et al, 2012, Mol. Ther., 20:948-953; Thess, A. et al., 2015, Mol Ther, 23(9):1456-1464;). Accordingly, multimeric and heteromultimeric VNAs with an estimated serum half-life of 1-2 days (with albumin-binding) are likely to benefit from being encoded by mRNA. As reported, the half-lives of VNA serum titers at one to three days after treatment were estimated to be, on average, 1.5-fold higher than from day three onward, even without target-specific mRNA optimization. (Mukherjee et al., 2014, PLoS ONE, 9e106422). In general, one to three days after treatment, both mRNA and protein half-lives contribute to the kinetics of serum titers, while after day three forward, the kinetics is almost exclusively determined by the properties of the expressed protein.

### Epitope Tags and Antibodies Thereto

In an embodiment, the VNA binding protein is a multimeric fusion protein engineered and produced to form a heteromultimeric complex that effectively binds to and neutralizes a disease agent or plurality of disease agents, in particular, the toxins of C. difficile and Botulinum microorganisms, e.g., C. difficile TcdA, C. difficile TcdB, a combination thereof, or Botulinum toxin A, Botulinum toxin B, or a combination thereof. In certain embodiments, the VHH amino acid sequences are coupled to epitope tag amino acid sequences or other sequences with linking amino acid sequences, e.g., EPKTPKPQGGGGSGGGGSGGGGSQGVQSQVQLVE (SEQ ID NO: 9) or EPKTPKPQ (SEQ ID NO: 10), as observed, for example, in FIGS. 1-4. In another embodiment, a dimerization agent that complexes peptide fragments each containing at least about 5 to 25 amino acids, 25 to 50 amino acids, 50 to 100 amino acids, 100 to 150 amino acids, and 150 amino acids to about 200 amino acids may be used. Multimerization agents and methods of using the agents for forming multimeric binding proteins can be found, for example, in U.S. Pat. Nos. 9,023,352, 8,349,326 and 7,763,445, each of which is incorporated by reference herein in its entirety.

In certain embodiments the multimeric VNA binding protein molecule includes a single tag (epitope tag) or multiple tags. For example, each multimeric binding protein includes at least one, or two or more epitope tags on each component binding region, which is termed a “monomer” unit of the binding protein molecule. In an embodiment, the binding molecule comprises no tag attached to the VHH monomer and/or linker components. In certain embodiments, the presence of an epitope tag operably linked, coupled, or fused to the VHH binding protein and/or the VHH binding region and bound by an anti-epitope tag antibody induces clearance of the toxin from the body, in particular, following binding of a multimeric VNA binding molecule to the toxin protein(s). In an embodiment, the binding of the one or more epitope tags in the binding molecule by anti-epitope tag antibody(ies) may synergistically induce clearance of the toxin from the body following binding by a VNA binding molecule.

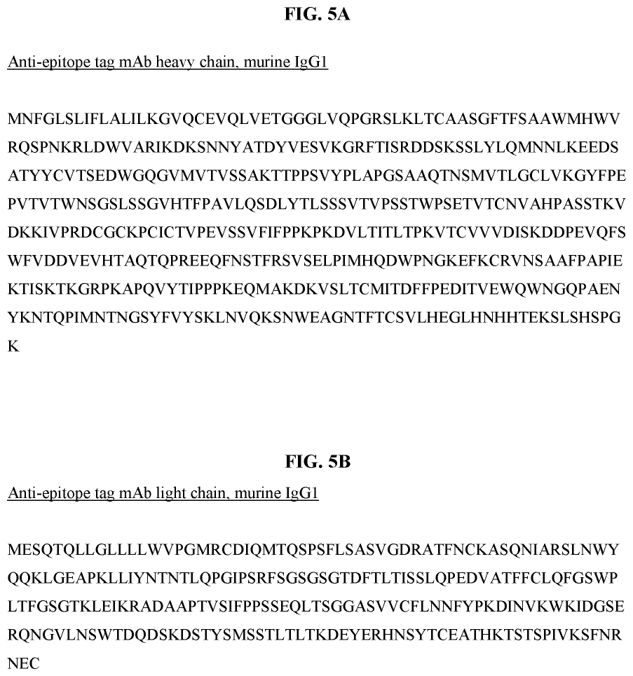

By way of example, an anti-tag (i.e., anti-epitope tag) antibody may be administered to a subject who is also treated with or administered a VNA binding molecule containing one or more epitope tags, or a pharmaceutical composition thereof. The anti-tag antibodies bind to the epitope tags of the VNA binding molecule, which, in turn, bind to one or more toxin proteins, thereby forming a complex that is rapidly cleared from the body (Sepulveda, J. et al., 2010, Infect. Immun., 78(2):756-763; Mukherjee, J. et al., 2012, PLoS ONE, 7(1): e29941. PMCID: PMC3253120; https://doi.org/10.1371/journal.pone.0029941). In an embodiment, an anti-epitope tag monoclonal antibody of a specific isotype, for example IgG1, may be provided to a subject who is also administered one or more multimeric VNA binding protein molecules as described herein, e.g., to enhance clearance from the body of the bound multimeric VNA binding molecule/target protein/anti-epitope tag antibody complex. The heavy and light chain sequences of an anti-epitope tag monoclonal antibody which binds to the epitope tag sequence contained in the multimeric VNA binding molecules described herein are provided as SEQ ID NOS: 20 and 21, respectively, in FIGS. 5A and 5B.

In an embodiment, the multimeric binding protein comprises two or more, e.g., four, VHH binding molecule components and two epitope tag sequences, with one tag attached to each monomer VHH molecule component. In an embodiment, an epitope tag is present at or near to both the amino and carboxy termini of the binding molecule. In another embodiment, the multimeric binding protein molecule comprises two or four VHH binding molecule components and one epitope tag sequence, which may be attached to one VHH monomer component at either the amino or carboxy terminus of the multimeric binding protein molecule. In an embodiment, a single VNA binding molecule, e.g., a recombinant multimeric binding protein as described herein that comprises two or four toxin-binding VHH monomer components joined by one or more linker sequences, that also comprises at least one, in particular, two, epitope tags is effective in binding and neutralizing toxin in a subject exposed to or infected by the relevant toxins. In an embodiment, recombinant multimeric VNA binding protein molecules having two or more identical or non-identical VHH binding protein components and administered to subjects either before or after contact with a toxin-producing microorganism provide effective anti-toxin treatment that are more efficacious than typical serum-based polyclonal antitoxins.

In certain embodiments, an anti-tag antibody may also effect or facilitate immunoglobulin effector functions. Anti-tag antibodies may include, for example, IgA, IgD, IgE, IgG, and IgM immunoglobulins and subtypes thereof. An immune response to an epitope tag included in a VNA binding molecule may involve the elicitation of specific monoclonal antibodies and/or polyclonal antibodies that specifically bind to the tag. Immunoglobulin effector functions may involve, for example, interaction(s) between the Fc portion of the immunoglobulin and receptors or other protein molecules in a subject or cells thereof. Depending on the immunoglobulin type, the effector functions result in clearance of the disease agent (e.g., excretion, degradation, lysis or phagocytosis). In an embodiment, an anti-tag antibody of one immunoglobulin effector type binds to the VNA binding protein molecules which comprise one or more epitope tags. In embodiments in which the VNA binding protein molecules comprise two or more epitope tags, an anti-tag antibody binds to each of the tags of the VNA binding protein molecule. In an embodiment, the VNA binding protein molecule comprises at least one epitope tag. In an embodiment, the VNA binding protein molecule comprises at least two identical epitope tags. In an embodiment, the VNA binding protein molecule comprises at least two different epitope tags.

In certain embodiments, the one or more multimeric VNA binding protein molecules as described herein are administered with a suitable anti-epitope tag antibody or a functional epitope tag binding fragment thereof.

Suitable methods of producing or isolating antibody fragments having the requisite binding specificity and affinity for binding to an epitope tag include for example, methods which select recombinant antibody from a library or by PCR (e.g., U.S. Pat. Nos. 5,455,030 and 7,745,587 each of which is incorporated by reference herein in its entirety).

Functional fragments of antibodies, including fragments of chimeric, humanized, primatized, veneered, or single chain antibodies, can also be produced. Functional fragments or portions of the foregoing antibodies include those which are reactive with the toxin protein. For example, antibody fragments capable of binding to the toxin protein or a portion thereof, include, but not limited to, scFvs, Fabs, VHHs, Fv, Fab, Fab′ and F(ab′)2. Such fragments can be produced by enzymatic cleavage or by recombinant techniques. For instance, papain or pepsin cleavage are used generate Fab or F(ab′)2 fragments, respectively. Antibody fragments are produced in a variety of truncated forms using antibody genes in which one or more stop codons has been introduced upstream of the natural stop site. For example, a chimeric gene encoding a F(ab′)₂ heavy chain peptide portion can be designed to include DNA sequences encoding the CH₁ peptide domain and hinge region of an immunoglobulin heavy chain.

### Pharmaceutical Compositions

The present invention features methods for treating or preventing pathologies and disease caused by toxins produced by C. difficile or Botulinum microorganisms following infection of a subject by these microorganisms. The methods include administering to a subject in need thereof an amount of one or more of the heteromultimeric VNA binding protein molecules, or such molecules administered with an anti-epitope tag antibody as described herein, that is effective to specifically bind and neutralize one or more of the bacterial toxins, e.g., C. difficile toxin A, toxin B, or a combination thereof, or Botulinum toxin A, toxin B, or a combination thereof. In an embodiment, the multimeric VNA binding protein molecules are provided or used in a pharmaceutical composition. In an embodiment, one or more multimeric VNA binding protein molecules specifically binds and neutralizes the toxin activity of C. difficile toxin A, toxin B, or a combination thereof, and are provided or used in a pharmaceutical composition.

Typically, a carrier or excipient is included in a composition as described herein, such as a pharmaceutically acceptable carrier or excipient, which includes, for example, sterile water, aqueous saline solution, aqueous buffered saline solutions, aqueous sucrose, dextrose, or mannose solutions, aqueous glycerol solutions, ethanol, calcium carbonate, albumin, starch, cellulose, silica gel, polyethylene glycol (PEG), dried skim milk, rice flour, magnesium stearate, and the like, or combinations thereof. The terms “pharmaceutically acceptable carrier” and a “carrier” refer to any generally acceptable excipient or drug delivery device that is relatively inert and non-toxic.

The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like. Such methods also include administering an adjuvant, such as an oil-in-water emulsion, a saponin, a cholesterol, a phospholipid, a CpG, a polysaccharide, variants thereof, and a combination thereof, with the composition of the invention. Optionally, a formulation for prophylactic administration may also contain one or more adjuvants for enhancing the effect of, or an immune response to, an antigen or immunogen, e.g., a multimeric VNA binding protein molecule as described herein. Suitable adjuvants include, without limitation, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59. In an embodiment, the multimeric VNA binding protein molecule is provided in a pharmaceutical composition.

The administration of a multimeric VNA binding protein molecule as described herein, or a pharmaceutical composition thereof, as a therapeutic for the treatment or prevention of disease or pathology caused by toxins produced by C. difficile or Botulinum infection may be by any suitable means that results in a concentration of the therapeutic that, combined with other components, if desired, is effective in ameliorating, reducing, eliminating, abating, or stabilizing disease, pathology, or the symptoms thereof in a subject. The therapeutic may be administered systemically, for example, formulated in a pharmaceutically-acceptable composition or buffer such as physiological saline.

Routes of administration include, for example and without limitation, subcutaneous, intravenous, intraperitoneal, intramuscular, intrathecal, intraperitoneal, or intradermal injections that provide continuous, sustained levels of the therapeutic in the subject. Other routes include, without limitation, gastrointestinal, esophageal, oral, rectal, intravaginal, etc.

The amount of the therapeutic to be administered varies depending upon the manner of administration, the age and body weight of the subject, and with the clinical symptoms of the bacterial infection or associated disease, pathology, or symptoms. Generally, amounts will be in the range of those used for other agents used in the treatment of disease or pathology associated with C. difficile or Botulinum infection, although in certain instances, lower amounts may be suitable because of the increased range of protection and treatment afforded by the multimeric VNA binding protein molecule as therapeutic. A composition is administered at a dosage that ameliorates, decreases, diminishes, abates, alleviates, or eliminates the effects of the bacterial (microorganism) infection or disease (e.g., CID or the symptoms thereof) as determined by a method known to one skilled in the art.

In embodiments, a therapeutic or prophylactic treatment agent may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the gut or gastrointestinal system; (v) formulations that allow for convenient dosing, such that doses are administered, for example, once every one or two weeks; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, one or more multimeric VNA binding protein molecules may be formulated with one or more additional components for administration to a subject in need, e.g., patients who have contracted C. difficile infection or Botulinum bacterial infections and suffer from the serious repercussions of toxin production by these microorganisms.

Any of a number of strategies can be pursued in order to obtain controlled release of a therapeutic agent in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

A pharmaceutical composition may be administered parenterally by injection, infusion, or implantation (subcutaneous, intravenous, intramuscular, intradermal, intraperitoneal, intrathecal, or the like) in dosage forms, formulations, or via suitable delivery devices or implants containing conventional, non-toxic pharmaceutically acceptable carriers and adjuvants. A pharmaceutical composition may also be provided by oral, bucal, topical (e.g., via powders, ointments, or drops), rectal, mucosal, sublingual, intracisternal, intravaginal, rectal, ocular, or intranasal administration. The formulation and preparation of such compositions are well known to those skilled in the art of pharmaceutical formulation. Formulations can be found in Remington: The Science and Practice of Pharmacy, noted supra.

Compositions for parenteral or oral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease, pathology, or symptom thereof, such as CID, C. difficile-associated diarrhea (CDAD), pseudomembranous colitis (PMC), bowel inflammation, enterocytic detachment, alteration, disruption, or elimination of natural intestinal microflora, and/or paralytic ileus caused by C. difficile infection, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, a pharmaceutical composition comprising an active therapeutic (e.g., one or more multimeric VNA binding protein molecules as described herein) is formulated for systemic delivery, intravenous delivery, e.g., intravenous injection, subcutaneous delivery, or local delivery (e.g., diffusion). To prepare such a composition, the suitable therapeutic(s) are dissolved or suspended in a parenterally acceptable liquid vehicle, excipient, or solvent. Among acceptable vehicles and solvents that may be employed are, for example, water; water adjusted to a suitable pH by the addition of an appropriate amount of hydrochloric acid, sodium hydroxide or a suitable buffer; 1,3-butanediol; Ringer's solution; and isotonic sodium chloride solution and dextrose solution. An aqueous formulation may also contain one or more preservatives (e.g., methyl, ethyl or n-propyl p-hydroxybenzoate). In cases in which a therapeutic agent is only sparingly or slightly soluble in water, a dissolution enhancing or solubilizing agent can be added, or the solvent may include 10-60% w/w of propylene glycol or the like.

In some embodiments, compositions comprising one or more multimeric VNA binding protein molecules are sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. In some embodiments, the multimeric VNA binding protein molecules are combined, where desired, with other active substances, e.g., enzyme inhibitors, to reduce metabolic degradation.

An effective amount of compositions can vary according to choice or type of the multimeric VNA binding protein molecule as described herein, the particular composition formulated, the mode of administration and the age, weight and physical health or overall condition of the patient, for example. In an embodiment, an effective amount of the VNA binding protein molecules and/or anti-epitope tag antibody is an amount which is capable of reducing one or more symptoms of the disease or pathology caused by the infectious agent/disease target. Dosages for a particular patient are determined by one of ordinary skill in the art using conventional considerations, (e.g. by means of an appropriate, conventional pharmacological protocol).

In certain embodiments, a composition includes one or more polynucleotide sequences that encode one or more of the multimeric VNA binding protein molecules as described herein. In an embodiment, a polynucleotide sequence encoding a multimeric VNA binding protein molecule is in the form of a DNA molecule or multimer. In some embodiments, the composition includes a plurality of nucleotide sequences each encoding a multimeric VNA binding protein molecule, or any combination of molecules described herein, such that the multimeric VNA binding protein molecule is expressed and produced in situ. In such compositions, a polynucleotide sequence is administered using any of a variety of delivery systems known to those of ordinary skill in the art, including eukaryotic, bacterial and viral vector nucleic acid expression systems. Suitable nucleic acid expression systems contain appropriate nucleotide sequences operably linked for expression in a patient (such as suitable promoter and termination signals). Bacterial delivery systems involve administration of a bacterium (such as Bacillus-Calmette-Guerrin) that expresses the polypeptide on its cell surface. In an embodiment, multimeric VNA binding protein molecule-encoding nucleic acid can be introduced using a viral expression system (e.g., vaccinia or other pox virus, retrovirus, or adenovirus), which uses a non-pathogenic (defective), replication competent virus. Techniques for incorporating nucleic acid (DNA) into such expression systems are well known to those of ordinary skill in the art. The nucleic acid (DNA) can also be “naked,” as described, for example, in Ulmer et al., 1993, Science, 259:1745-1749 and as reviewed by Cohen, 1993, Science 259:1691-1692. The uptake of naked DNA can be increased by coating the DNA onto biodegradable beads, which are efficiently transported into recipient cells.

### Therapeutic Methods

Methods of treating disease, conditions, pathology and/or symptoms thereof associated with C. difficile infection are provided. The methods comprise administering a therapeutically effective amount of one or more multimeric VNA binding protein molecules as described herein, or a pharmaceutical composition comprising such one or more molecules to a subject (e.g., a mammal such as a human). In an embodiment, a method of treating a subject suffering from or susceptible to CDI, C. difficile-associated diarrhea (CDAD), pseudomembranous colitis (PMC), bowel inflammation, enterocytic detachment, alteration, disruption, or elimination of natural intestinal microflora, and/or paralytic ileus caused by C. difficile infection, or to botulinum disease or wounds resulting from Botulinum infection or a symptom thereof. The method includes the step of administering to the mammal a therapeutic amount of one or more multimeric VNA binding protein molecules as described herein sufficient to treat the disease, illness, condition, disorder and/or symptom thereof, under conditions such that the disease or disorder is treated.

The therapeutic methods of the invention (which include prophylactic treatment) in general comprise administration of a therapeutically effective amount of one or more or the multimeric VNA binding protein molecules as described herein, to a subject or patient in need thereof. A subject or patient is meant to include an animal, particularly a mammal, and more particularly, a human. Such one or more multimeric VNA binding protein molecules used as treatment will be suitably administered to subjects or patients suffering from, having, susceptible to, or at risk for a disease, disorder, or symptom thereof caused by or associated with infection by C. difficile or C. botulinum and their produced toxin proteins. Determination of patients who are “at risk” can be made by any objective or subjective determination obtained by the use of a diagnostic test or based upon the opinion of a patient or a health care provider (e.g., genetic test, enzyme or protein marker, family history, and the like). The one or more multimeric VNA binding protein molecules as described herein may be also used in the treatment of any other disorders in which the one or more target protein toxins may be implicated.

The methods herein include administering to the subject (including a subject identified as in need of such treatment) an effective amount of one or more multimeric VNA binding protein molecules as described herein, or a composition described herein to produce such effect. Identifying a subject in need of such treatment can be in the judgment of a subject himself or herself, or of a health care/medical professional and can be subjective (e.g., opinion) or objective (e.g., measurable or quantifiable by a test or diagnostic method).

### Methods of Use

In an aspect, a method of monitoring treatment progress is provided. The method includes determining a level of toxin protein as an indicator of disease or infection in a subject suffering from or susceptible to infection by, or disease or illness associated with infection by C. difficile or C. botulinum bacteria, in which the subject has been administered a therapeutic amount of one or more of the multimeric VNA binding protein molecules as described herein sufficient to treat the disease or symptoms thereof. According to the method, the level of the toxin protein(s) (which serves as a marker of infection or disease) is detected, measured, or quantified in a biological sample obtained from the subject relative to known levels of the same toxin protein(s) in healthy normal controls and/or in other afflicted patients to establish the subject's treatment progress, disease progress, or disease status. In embodiments, the levels of toxin protein(s) in the subject's sample are measured or quantified at one or more later time points (following the previous measurements), relative to the levels previously detected or measured in the subject, and/or relative to the levels in normal/healthy subjects or in other afflicted patient controls so as to monitor the course of disease or the efficacy of the therapy. In certain embodiments, a pre-treatment level of toxin protein(s) in the subject is determined prior to beginning treatment according to the method; this pre-treatment level of toxin protein(s) can then be compared to the level of the toxin protein(s) in the subject after the treatment commences to monitor or determine the efficacy of the treatment.

### Methods of Delivery

In an embodiment, a single multimeric VNA binding protein molecule can be administered to a subject in need of treatment for intoxication by a toxin protein, such as TcdA, TcdB, or a combination thereof. In an embodiment, a mixture of multimeric VNA binding protein molecules can be administered to a subject in need of treatment, depending on the type of bacterial infection with which a subject is afflicted. In an embodiment, the multimeric VNA binding protein molecule(s) may include one or more epitope tag sequences to which anti-epitope tag antibody(ies) specifically bind. In another embodiment in which the multimeric VNA binding protein molecule(s) administered to a subject includes one or more epitope tag sequences, a specific anti-epitope tag antibody can also be administered to the subject in need of treatment.

In some embodiments, the administration of two or more multimeric VNA binding protein molecule(s) may increase the effectiveness of the therapy and reduce the severity of one or more negative symptoms related to exposure of the subject to the protein toxin target. In an embodiment, administering to a subject a multimeric VNA binding protein molecule that includes two or epitope tag sequences may result in improved therapy, treatment, or protection against disease caused by a toxin protein target, e.g., Botulinum toxin A. In an embodiment, all of the epitope tag sites of a multimeric VNA binding protein molecule are bound by specific anti-tag antibody or by one type of anti-tag antibody. In embodiments, antibody therapeutic proteins or an antibody therapeutic preparation refers to one or more compositions that include at least one multimeric VNA binding protein molecule as described herein and, optionally, at least one anti-epitope tag antibody. In embodiments, the compositions or preparations comprising one or more multimeric VNA binding protein molecules as described herein contain additional reagents or components, including carriers as described supra.

The administration of the one or more multimeric VNA binding protein molecules as described herein and the administration of one or more anti-epitope tag antibodies may be performed simultaneously or sequentially in time. In an embodiment, the one or more multimeric VNA binding protein molecule is administered before, after, or at the same time as the administration of another multimeric VNA binding protein molecule or the anti-tag antibody, provided that the multimeric VNA binding protein molecule and/or the anti-tag antibody(ies) are administered close enough in time to have the desired effect (e.g., before the multimeric VNA binding protein molecules have been cleared by the body). Accordingly, “co-administration” embraces the administration of a multimeric VNA binding protein molecule and a subsequent multimeric VNA binding protein molecule or the anti-tag antibody at time points that will achieve effective treatment of the disease, such as reduction in the level of toxin protein(s) and disease and symptoms associated with the presence of the toxin. The described methods are not limited by time intervals between which the multimeric VNA binding protein molecules and/or the anti-tag antibody(ies) are administered; provided that these agents, or compositions containing these agents, are administered close enough in time to produce or achieve the desired effect. In an embodiment, only a multimeric VNA binding protein molecule is administered. In another embodiment, a multimeric VNA binding protein molecule and an anti-epitope tag antibody are premixed and administered together, or a not premixed but are co-administered within minutes of each other. In other embodiments, the multimeric VNA binding protein molecule and anti-epitope tag antibody(ies) are co-administered with other medications, drugs, compounds, or compositions suitable for treating the disease agent.

In yet other embodiments, the multimeric VNA binding protein molecule is administered to a subject prior to the potential risk of exposure to infection by a toxin producing microorganism (e.g., C. difficile or C. botulinum), or prior to exposure to the toxin protein target of the multimeric VNA binding protein molecule (e.g., TcdA, TcdB, or BtA) in order to protect a subject from disease and symptoms of the toxin protein target. For example, the multimeric VNA binding protein molecule and/or anti-epitope tag antibody (“clearing antibody) is administered minutes, hours or days prior to the risk of exposure to infecting bacteria and/or disease-causing toxin. Alternatively, the multimeric VNA binding protein molecule is administered concomitantly with the risk of exposure of a subject to the infectious bacteria and production of the toxin protein target, or slightly after the risk of exposure. For example, the multimeric VNA binding protein molecule is administered to a subject at the moment that the subject contacts, enters, or passes through an environment (e.g., room, hallway, building, and field) containing a risk of exposure to a disease agent.

The methods of the present invention provide treating or protecting a subject from intoxication and disease (and the symptoms thereof) caused by one or more toxin proteins produced by a bacteria that have infected the subject. In accordance with the method, one or more multimeric VNA binding protein molecules and, optionally, an anti-tag antibody(ies) as described herein are administered to the affected or at risk subject. Administration ameliorates, reduces, or alleviates the severity of one or more the symptoms of the bacterial-induced disease or condition. The presence, absence, or severity of symptoms is measured, for example, using physical examination, tests and diagnostic procedures known and practiced in the art. In certain embodiments, the presence, absence and/or level of the bacteria and/or one or more produced protein toxins are measured using methods known and employed in the art. Symptoms or levels of the toxin protein(s) can be measured at one or more time points (e.g., before, during and after treatment, or any combination thereof) during the course of treatment with one or more multimeric VNA binding protein molecules to determine if the treatment is effective. A decrease, reduction, or no change in the levels of the toxin protein(s), or in the severity of symptoms associated therewith indicates that treatment is effective, and an increase in the level of the toxin protein(s), or in the severity of symptoms in a subject indicates that treatment is not effective. In various embodiments, the symptoms and levels of disease agents, such as toxin protein(s), are measured using methods known and employed in the art. Symptoms that are monitored in certain embodiments may generally include fever, pain including headache, joint pain, muscular pain, difficulty breathing, lethargy, and impaired mobility, appetite and unresponsiveness. Specific symptoms that are monitored in subjects with C. difficile infection may include watery diarrhea, cramping/tenderness (mild symptoms); severe watery diarrhea, colitis, severe abdominal cramping/tenderness, rapid heart rate, fever, blood in stool, dehydration, loss of appetite and weight, swollen abdomen, kidney failure and increase leukocyte count (severe symptoms). Specific symptoms that are monitored in subjects with C. botulinum infection may include difficulty swallowing or speaking, dry mouth, facial weakness, blurred or double vision, drooping eyelids, difficulty breathing, nausea, vomiting and abdominal cramping and paralysis. Treatment or protection from intoxication by one or more toxin proteins is assessed as increased survival and reduction, alleviation, or prevention of symptoms. Methods, compositions and kits involving the use of the multimeric VNA binding protein molecules as described herein decrease and alleviate the symptoms of the disease target agent (one or more toxin proteins) and also improve survival from exposure to the bacteria that produce the toxins and the toxin proteins themselves.

In an embodiment, a polynucleotide encoding a multimeric, toxin-binding VNA molecule as described herein, in particular, mRNA, in the form of lipid nanoparticles is used to deliver the binding molecules and produce effective and long-lasting antibody titers in subjects who are passively immunized with the mRNA-nanoparticles. In a particular embodiment, the mRNA, which is otherwise unmodified, may be codon optimized to afford efficient expression of the toxin-binding multimeric VNA molecules from the transcribed mRNA. In addition, exogenous mRNA has been reported to have the ability to instruct cells to produce VNAs, as well as other types of antibodies. See, M. Thran et al., 2017, EMBO Mol. Medicine, online publication no. DOI 10.15252/emmm.201707678.

The advantages of using mRNA for passive immunization are appreciated by those in the art. (See, M. Thran et al., Id.). By way of example, exogenous mRNA was demonstrated to direct protein expression in vivo, which has led to the use of mRNA as a promising drug platform technology (J. A. Wolff et al., 1990, Science, 247: 1465-1468; G. F. Jirikowski et al, 1992, Science, 255: 996-998). Several studies have demonstrated the utility of mRNA as the basis of vaccines to promote prophylactic protection from cancer and infectious diseases (I. Hoerr et al, 2000; Eur J Immunol 30: 1-7; B. Petsch et al, 2012, Nat Biotechnol, 30: 1210-1216). Primates and domestic pigs that were treated with an mRNA-encoded hormone produced physiological responses, thus demonstrating that mRNA therapy for large animals is feasible and effective. (Thess et al, 2015, Mol Ther, 23: 1456-1464). mRNA-based approaches for therapeutics may be safer and more cost effective compared with DNA-based approaches. Because mRNA does not integrate into a host's DNA and is more transient in nature, mRNA-based protein expression is considered to be easier to control of protein expression. However, mRNA's more transient expression may also limit its bioavailability in certain types of treatments, although high levels of expression (high titers) of encoded proteins, e.g., toxin binding VNA proteins as described herein, in serum following mRNA immunization have been demonstrated and are highly encouraging.

### Kits

The invention provides kits for the treatment or prevention of an infection or disease caused by or associated with C. difficile or C. botulinum bacterial pathogens. In some embodiments, the kit includes an effective amount of one or more multimeric VNA binding protein molecules as described herein, in unit dosage form. In an embodiment, the kit further contains an anti-epitope tag antibody, in unit dosage form. In other embodiments, the kit includes a therapeutic or prophylactic composition containing an effective amount of one or more multimeric VNA binding protein molecules in unit dosage form. In still other embodiments, the kit includes a therapeutic or prophylactic composition containing an effective amount of one or more multimeric VNA binding protein molecules and an anti-epitope tag antibody, in unit dosage form. In some embodiments, the kit comprises a device (e.g., an automated or implantable device for subcutaneous delivery; an implantable drug-eluting device, or a nebulizer or metered-dose inhaler) for dispersal of the composition or a sterile container which contains a pharmaceutical composition. Non-limiting examples of containers include boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition of the invention is provided together with instructions for administering the pharmaceutical composition containing one or more multimeric VNA binding protein molecules or one or more multimeric VNA binding protein molecules and an anti-epitope tag antibody to a subject having or at risk of contracting or developing an infection or disease or pathology, and/or the symptoms thereof, associated with infection by C. difficile or C. botulinum bacteria. The instructions will generally include information about the use of the composition for the treatment or prevention of an infection and intoxication by the C. difficile or C. botulinum bacteria and the toxin proteins that they produce. In other embodiments, the instructions include at least one of the following: description of the therapeutic/prophylactic agent; dosage schedule and administration for treatment or prevention of infection, disease or symptoms thereof caused by one or more of C. difficile or C. botulinum bacteria and/or the toxin proteins that they produce; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another aspect, a kit is provided for treating a subject exposed to, intoxication by, or at risk for exposure to or intoxication by a disease agent, such as C. difficile toxin A, toxin B, or both toxins A and B, or to C. botulinum toxin A or toxin B, or both toxins A and B, including: a pharmaceutical composition for treating a subject at risk for exposure to or exposed to the disease agent, the pharmaceutical composition including: at least one recombinant heteromultimeric VNA binding protein comprising a plurality of binding regions (VHH binding protein components) as described herein, such that the binding regions are identical or non-identical, and each binding region specifically binds a non-overlapping portion of the disease agent, such that the VNA binding protein neutralizes the disease agent, thereby treating the subject for exposure to the disease agent; a container; and, instructions for use. In various embodiments, the instructions for use include instructions for a method for treating a subject at risk for exposure to, exposed to, or intoxicated by the disease agent using the pharmaceutical composition.

In an embodiment, the multimeric VNA binding protein molecule includes at least one linker or linker amino acid sequence. In a specific embodiment, the linker includes amino acid sequence GGGGS (SEQ ID NO: 4), or GGGGSGGGGSGGGGS (SEQ ID NO: 5), or a functional portion thereof.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

### Example 1

### Materials and Methods

### Preparation of DNA

Synthetic DNA is prepared in which polynucleotides coding for the component molecules of the heteromultimeric polypeptides as described herein and as shown, for example, in FIGS. 1-4 are linked together, each separated by DNA encoding a flexible spacer, e.g., repeated GGGGS sequence, to encode VNA polypeptides comprising two or four toxin-binding VHH molecules per multimeric polypeptide. A procedure for making the multimeric polypeptides comprising linked or coupled component molecules may be found, for example, in Yang, Z. et al., 2014, J Infect. Dis., doi:10.1093/infdis/jiu196. In some cases, the multimeric DNA is inserted into an expression vector, such as pET32b, in fusion with E. coli thioredoxin (Trx), Trx-VNA, for example, as described by J. M. Tremblay (2013, Infec. Immun., 81:4592-4603) to create a Trx/VNA multimeric polypeptide expression plasmid.

### Protein Purification

The VNA multimeric polypeptide expression plasmid (pET32b) is transformed into Rosetta-gami (DE3) E. coli and fermentation cultures of the transformed bacteria is performed as follows: bacterial cells are cultured in 20 L of LB medium with 100 μg/ml ampicillin, 34 μg/ml chloramphenicol and incubated at 15° C. Expression is induced with 1 mM IPTG at OD₆₀₀=0.6 for 20 hours (J. M. Tremblay et al., 2010, Toxicon., 56:990-998). Protein are captured by Ni-affinity chromatography and eluted with 0.5M Imidazole gradient at pH 7.5. Protein are further purified by gel filtration chromatography (HiLoad Superdex 200 26/60, GE Life Sciences) and eluted with elution buffer containing 20 mM HEPES, pH 7.8, 200 mM NaCl, 1 mM DTT and 1 mM EDTA. The protein is expected to elute as a monomer from the gel filtration column. Recombinant protein is visualized by SDS-PAGE/Coomassie blue staining and blotted on a Western blot using anti-E-tag antibody (BETHYL) at 1:5,000 dilution. In some experiments, protein is treated for endotoxin removal by a Triton X114 phase partitioning method. Final endotoxin concentration in endotoxin-free preparation should be below 0.01EU/mg as determined by PYROGENE™ recombinant Factor C assay (Lonza Group Ltd, Basel Switzerland). In some cases, fermentation, purification, dialysis and endotoxin removal is performed by ARVYS Proteins Inc, Trumbull, Conn.

mRNA Sequence Engineering

The codons of the open reading frame may be adapted in order to improve translation and half-life of VNA-encoding mRNA, as described by Thess et al., 2015, Mol. Therapy, 23(9):1456-1464. As described, only the most GC-rich codons are used for each amino acid. To provide an optimized open reading frame (ORF) sequence with an optimal combination of untranslated sequences, the ORF sequence is subjected to a screening process applying preselected sequences. For the preselection of efficacious regulatory sequences, various biological sources are screened for potent enhancer and stabilizer elements. For example, sequence-engineered mRNAs can include a cap, an optimized open reading frame (i.e., a GC-enriched ORF), a 5′-UTR from HSD17B4 (hydroxysteroid (17-β) dehydrogenase 4), a 3′-UTR from ALB (albumin), and a polyA plus a histone stem loop. Examples of optimized mRNA sequences are shown in A. Thess et al., 2015, Mol. Therapy, 23(9):1456-1464). Sequence engineered (optimized) mRNA sequences may be molecularly cloned into expression/production vectors using flanking cloning sites as routinely practiced in the art.

mRNA Synthesis

Briefly, linearized plasmid harboring the sequence of interest downstream of a T7 promoter is transcribed using T7 RNA polymerase (Thermo Scientific, Braunschweig, Germany), (Thess et al., 2015, Id.). For capping of the RNA, m7G capping and 2′-O-methyltransferase kits (CellScript, Madison, Wis.) are used. 100% replacement is used for mRNAs that harbor chemically-modified nucleosides. All mRNAs lacking modified nucleosides (sequence-engineered or not) as well as all nucleoside-modified mRNAs are purified according to the same protocol by reversed-phase chromatography using a PLRP-S stationary phase and an acetonitrile gradient in a triethylammonium acetate buffer. A detailed protocol has been described by K.

mRNA Formulation for In Vivo Application

For intraperitoneal administration, mRNAs may be formulated with TransIT-mRNA (Mirus Bio, Madison, Wis.) according to a protocol described by K. Intravenous administration of mRNA to macaques or pigs may be conducted using mRNA encapsulated in lipid nanoparticles (LNPs). By way of specific example, Acuitas Therapeutics (Vancouver, Canada) has developed an mRNA delivery platform from technology which enables siRNA-dependent hepatic gene silencing across species in rodents, nonhuman primates and humans (M. LNPs are prepared using a self-assembly process in which an aqueous solution of mRNA at pH 4.0 is rapidly mixed with a solution of lipids dissolved in ethanol (. LNPs suitable for use may be similar in composition to those reported previously, which contain an ionizable cationic lipid/phosphatidylcholine/cholesterol/PEG-lipid (50:10:38.5:1.5 mol/mol), encapsulated RNA-to-total lipid ratio of ˜0.05 (wt/wt) and a diameter of ˜80 nm (M. at blood pH, LNPs exhibit a net neutral surface charge, but become positively charged in acidified endosomes following ApoE-mediated endocytosis by hepatocytes in vivo, resulting in endosome disruption and release of mRNA into the cytoplasm. (M. Jayaraman et al., 2012, Id.; M. J. Hope, 2014, Thess et al., 2015, Id.).

mRNA Encoding VNA Toxin Binding Molecules

The VNA molecules described herein are encoded by RNA polynucleotide, in particular, mRNA, for use in mRNA-mediated VNA binding molecule expression and in the treatment and protection of recipient subjects against intoxication by toxins (e.g., bacterial toxins such as C. difficile toxin A (TcdA), C. difficile toxin B (TcdB), or Botulinum A toxin). The design and synthesis of monoclonal antibody-encoding and single chain antibody-encoding mRNA sequences have been described (Thess, A. et al., 2015, Mol Ther, 23(9):1456-1464). In brief, mRNA encoding the VNAs described herein contains a 5′ cap structure, a 5′ UTR, an open reading frame, a 3′ UTR and poly-A sequence followed by a C30 stretch and a histone stem loop sequence (Thess, A. et al., 2015, Mol Ther, 23(9):1456-1464; Thran, M. et al., 2017, EMBO Molecular Medicine, DOI: 10.15252/emmm.201707678). Sequences are codon-optimized for human use and optimally do not include chemically modified bases. mRNAs encoding the VNAs as described herein can be capped enzymatically using ScriptCap 2′-O-methyltransferase (Biozym, Cat. 150360) and ScriptCap m7G Capping System (Biozym, Cat. 150355). For in vivo studies, mRNAs were further polyadenylated using A-Plus Poly(A) Polymerase Tailing Kit (Biozym, Cat. 150491). Capping and polyadenylation were carried out according to manufacturer's specifications.

### RNA Formulation

The formulation of lipid nanoparticles containing mRNA-encoding VNA binding molecules (mRNA-LNP) is described in Thess et al., 2015, Mol Ther, 23(9):1456-1464 and Thran, M. et al., 2017, EMBO Molecular Medicine, DOI: 10.15252/emmm.201707678. For injection into a recipient, mRNA-LNP are diluted in phosphate-buffered saline pH 7.4.

### Cell Transfections

For in vitro transfection of cells (e.g., BHK cells, Examples 2 and 3), RNAs were complexed with Lipofectamine 2000 (Life Technologies, Darmstadt, Germany) at 1.5 μl/μg of mRNA for BHK cells (or at 1 μl/μg for HepG2 cells) and were transfected into cells using standard procedures practiced in the art. To analyze the expression of a VNA binding molecule, 400,000 BHK cells were seeded in 6-well plates one day before transfection. Cells were transfected with 5 μg of VNA-encoding RNAs. Approximately 2 hours after transfection, transfection mix was replaced by 1.5 ml of serum-free freestyle 293 medium (Thermo Scientific Cat. 12338018). Cells were grown for approximately 48 hours, and supernatants were harvested and centrifuged to remove cell debris. After centrifugation, the supernatants were used for analyses, such as immunoassay (ELISA), (FIG. 2), or neutralization assays (FIG. 3). For preparation of cell lysates, the cells were incubated for 5 minutes with Laemmli buffer and then were collected.

### Western Blot Analysis

For Western blot experiments, pooled triplicates of equal amounts of cell lysates or supernatants are loaded and 12% SDS Tris-glycine gels are used. Proteins are transferred to a nitrocellulose membrane (Odyssey nitrocellulose membrane 0.22 μm, Li-COR Biosciences, Cat. 926-31092) and blocked in 5% skimmed milk in TBST buffer (TBS containing 0.1% TWEEN®-20 from Sigma, Cat. P2287). Membranes are first incubated with rabbit anti-α/β-tubulin 1:1,000 (New England Biolabs Cat. 2148S) in 0.5% skimmed milk in TBST for 1 hour. The membranes were washed three times (10 minutes per wash) in TBST. To al, eukary binding molecules, the following primary antibodies are used: mouse anti-β actin antibody (Abcam, Cat. ab6276) at a 1:20,000 dilution, and rabbit anti-E-tag antibody (Bethyl, A190-133A) at a 1:10,000 dilution. The secondary antibodies used are goat anti-mouse IgG (H+L) IRDye® 680RD (Li-COR, Cat. 926-32210) at a at 1:10,000 dilution, and goat anti-rabbit IgG (H+L) IRDye® 800CW (Li-COR, Cat. 926-32211) at a 1:15,000 dilution in 0.5% skimmed milk and membranes are incubated in TBST for 1 hour. Immediately before detecting bands on the Western blot, all membranes are washed three times in TBST for 10 minutes and stored in TBS lacking TWEEN®-20 until analysis. Protein detection and image processing are carried out in an Odyssey® CLx Imaging system and LI-COR's Image Studio version 5.2.5 according to manufacturer's instructions.

### VNA Functional Testing

VNA-encoding mRNAs were transfected into BHK cells as described above, and supernatants were collected at 48 or 72 hours. Collected supernatants were tested for their ability to bind specific toxins (see, Example 2 infra) and to inhibit the effects of the respective toxins in cell-based assays (see, Example 3, infra).

### Animal Experiments

Animal experiments involving mRNA-encoded VNAs are conducted at the Department of Infectious Disease and Global Health, Tufts Cummings School of Veterinary Medicine (North Grafton, USA) in conformance with Tufts University IACUC Protocol #G2016-74. Six- to eight-week-old female CD1 mice (Charles River Labs, Wilmington, USA) are randomized based on body weight and receive single intravenous injections of LNP-formulated mRNA into the tail vein. Blood is sampled by retro-orbital bleeding at defined times, and VNA accumulation in sera is measured as described by J. M. Tremblay et al., 2013, Infec. Immun., 81:4592-4603; Mukherjee et al., 2014, PLoS ONE 9e106422. In all studies, animals are housed under standard and humane conditions with a standard commercial rodent diet and tap water provided to the animals ad libitum.

### Computational Analysis

In general, data are analyzed using GraphPad Prism software version 6. All error bars refer to standard deviations. ELISA data are analyzed using nonlinear regression.

### Enzyme Linked Immunosorbent Assay (ELISA)

EIA/RIA 96 well high binding plates (Corning Costar) coated with 0.5-5 μg/ml of recombinantly produced VNA molecules, e.g., rTcdA or rTcdB or rTcdA+rTcdB, overnight at 4° C. were used for immuno-binding assays (ELISA). Plates were washed 3 times with 1×PBS+0.1% Tween, followed by washing 3 times with 1×PBS. Washed plates were blocked (4-5% non-fat dry milk in 1×PBS+0.1% Tween) for 1 hour at room temperature (RT) with rocking. Serially diluted (1:5) binding molecules, VNA-TcdA targeting C. difficile toxin A; VNA-TcdB targeting C. difficile toxin B, and VNA2-Tcd targeting both C. difficile toxin A and C. difficile toxin B, diluted in blocking solution were incubated for 1 hour at RT with rocking and washed as above. Equivalent control samples were spiked with a known amount of VNA2-Tcd for use as an internal standard. Binding of VNAs to recombinant toxin coating the wells was detected using anti-O-tag antibody detection with peroxidase labeled antibodies and assessed by an ELISA reader at A450 nm. Illustratively, the plates were incubated with goat anti-O-tag-HRP conjugated antibody (Bethyl labs) diluted 1:5000 in blocking solution for 1 hour at RT with rocking and were washed as above before adding TMB microwell peroxidase substrate (KPL) to develop (incubated for 10-40 min). Development was stopped with 1M H₂SO₄ and the plates were read at 450 nm on an ELx808 Ultra Microplate Reader (Bio-Tek instruments), (Mukherjee, J. et al., 2012, PloS ONE 7:e29941). VNA2-Tcd levels in unknown samples were determined by comparison of their signals to those of internal standards as previously described (Mukherjee, J. et al., 2014, PLoS One 9:e106422; Sheoran, A S et al., 2015, Infect Immun, 83:286-291; Moayeri, M. et al., 2016, Clin Vaccine Immunol, doi:10.1128/cvi.00611-15; Sponseller, J K et al., 2014, J Infect Dis, doi:10.1093/infdis/jiu605; Tzipori, S. et al., 1995, Infect Immun, 63:3621-3627). (See, e.g., Example 2).

### Neutralization Assay

Vero cells (ATCC) at a concentration of 2.4×10⁴ cells/100 μl of medium (DMEM high glucose+1 mM sodium pyruvate, 2 mM L-glutamine, 50 U/ml and 50 μg/ml Pen/Strep pH 7.4 (HyClone)) were plated in 96-well plates overnight for 90-95% confluency, prior to addition of VNA2-Tcd added in serial dilutions (in media) (1:5) or from 100 μg/ml-1.0 fg/ml and toxic levels of TcdA or TcdB, such as 2 ng-12.5 ng/ml TcdA and 0.25-2 ng/ml TcdB, or both TcdA and TcdB, in a 24 hour cytotoxicity/cell rounding assay (Yang, Z. et al., 2014, J Infect Disease, doi:10.1093/infdis/jiu196). After 24 hours, the plates were assessed for toxicity to the cells by quantifying the percent (%) of cells that became rounded in each of the wells. (See, e.g., Example 3).

### Mouse Systemic Toxin Challenge

For in vivo studies using mice, 6 week old C57BL/6 female mice are intraperitoneally (IP) injected with a single dose of VNA, e.g., VNA2-Tcd (50 μg/mouse), 1 hour prior to IP injection of TcdA (100 ng/mouse), TcdB (200 ng/mouse), or TcdA+TcdB (100 ng and 200 ng respectively). Mice are monitored for signs and symptoms of toxemia (including; lethargy, depression, anorexia, dehydration, ruffled coat, and hunched posture). Moribund mice were euthanized following IACUC-approved removal criteria.

### Mouse CDI Challenge

In vivo experiments using mice are conducted to mimic the human condition of C. difficile infection (CDI) and disease and to facilitate colonization with C. difficile. For these experiments, ten 6 week old, C57BL/6 female mice receive filter sterilized antibiotics (kanamycin, gentamycin, colistin, metranidozole, and vancomycin) in drinking water for 5 days followed by 2 days of water alone. After 2 days of drinking the supplemented water, the mice receive one (100 μl) intraperitoneal (IP) injection of clindamycin (2 mg/ml). One day later mice are orally challenged (Chen, X. et al., 2008, Gastroenterology, 135:1984-1992) with 10⁶ spores of an NAPI/027/BI C. difficile strain, designated strain UK6 (Killgore, G. et al., 2008, J. Clin Microbiol, 46:431-437) only (control group), or are inoculated with spores and administered VNA2-Tcd (25-50 μg/mouse) at 4, 24 and 48 hrs post-challenge (treated group). Blood is collected at 72, 96 and 120 hours post-challenge to determine VNA titers.

### Hamster CDI Challenge

In vivo experiments using hamsters are conducted to mimic the human condition of C. difficile infection and disease and to facilitate colonization with C. difficile. For these experiments, male Golden Syrian hamsters (110-135 g) are administered clindamycin (30 mg/kg) via oral gavage for 5 days prior to oral inoculation with 1000 C. difficile strain UK6 spores. Infected control hamsters are administered clindamycin, are inoculated with UK6 spores and are given sterile PBS, by intraperitoneal (IP) administration, 2 times per day for the duration of the experiment. VNA2-Tcd treated hamsters are administered clindamycin, inoculated with spores and are given purified VNA2-Tcd (1 mg/kg), by IP administration, 2 times a day for the duration of the experiment. A blood sample is collected at time of euthanasia for detection of VNA2-Tcd in serum. Necropsies are performed on euthanized animals and tissues are collected for histopathologic examination.

### Pig CDI Challenge

Pigs have been demonstrated to mimic C. difficile infection, colonization and disease as experienced by humans. For in vivo experiments using pigs, thirty gnotobiotic piglets are derived via Caesarean section and maintained in sterile isolators for the duration of the experiment (Tzipori, S. et al., 1995, Infect Immun, 63:3621-3627). Five groups of piglets are orally inoculated with 10⁶ C. difficile UK6 spores (group 1-5) and group 6 was the uninfected control group. Group 1 (n=3) receives VNA2-Tcd (1 mg/pig) 4 hours prior to, and Group 2 (n=3) 18 hours post oral inoculation with spores. After the initial dose, the treated groups receive 2 doses of VNA2-Tcd (1 mg/pig) per day either via IP or intra muscular (IM) administration for the duration of the experiment. The Ad/VNA2-Tcd treated group (Group 3; n=9) is given 1.0×10¹¹ viral particles by IV administration one day prior to oral inoculation with 10⁶ C. difficile UK6 spores and 3 days post infection. Group 4 (n=6) receives VNA-Tcd buffer as control, given 4 hours prior to oral inoculation with 10⁶ C. difficile UK6 spores and at 24 hour post inoculation, and then every 12 hours until the termination of the experiment. Group 5 is given control adenovirus expressing an unrelated VNA (n=6), (1.0×10¹¹viral particles) by IV administration one day prior to oral inoculation with 10⁶ C. difficile UK6 spores and 3 days post infection. Group 6 (n=3) is uninfected. Fecal samples are collected from all piglets for bacterial culture, and blood samples are collected 1-3 times (when possible) during the experiment and at the time of euthanasia to determine VNA titers. Necropsies are performed on all animals and tissues are collected for histopathologic examination.

### Histology

Tissue samples from C. difficile colonized animals are collected during necropsy and preserved in 10% neutral buffered formalin. Formalin fixed samples are embedded in paraffin, sectioned at 5 μm, and stained using hematoxylin and eosin using routine histochemical techniques at TCSVM Histopathology Service Laboratory (http://vet.tufts.edu/histology-service/). Light microscopic examination and lesion evaluation are performed by a board-certified veterinary pathologist (GB) with results reported for severity (minimal, mild, moderate, marked), epithelial ulceration, luminal contents, and quantification (Sponseller, J K et al., 2014, J Infect Dis, doi:10.1093/infdis/jiu605). Briefly, a quantitative assessment of colitis severity is performed by counting neutrophilic foci in colon sections from each sample. Foci are observed between colonic crypts in the lamina propria in 10 random fields with ×20 magnification.

### Example 2

Specific Binding of Toxins by VNA Binding Molecules Targeting C. difficile Toxins A, B, or A and B

To assess specific binding to toxins by the VNA binding molecules described herein, BHK cells were transfected with nanoparticles comprising RNA encoding the binding molecules, such as the VNA binding molecules of FIGS. 1-3 herein. The supernatants were collected at 48 hours post-transfection. Titers of VNA in the supernatants were determined in triplicate. The collected supernatants were tested by standard dilution immunoassay (ELISA) methods as described in Example 1 using ELISA plates coated with either recombinant TcdA or TcdB toxins and using serial dilutions (1:5) of conditioned medium. Binding of VNAs to wells was detected using anti-O-tag antibody detection with peroxidase labeled antibodies and assessed by ELISA reader at A450. Medium alone showed no binding to the plate-bound toxins, while conditioned medium expressing the different VNAs clearly showed binding to the appropriate toxin down to a dilution of greater than 1:1000. In some experiments, supernatants from BHK cells transfected with an irrelevant VNA were used as mock control.

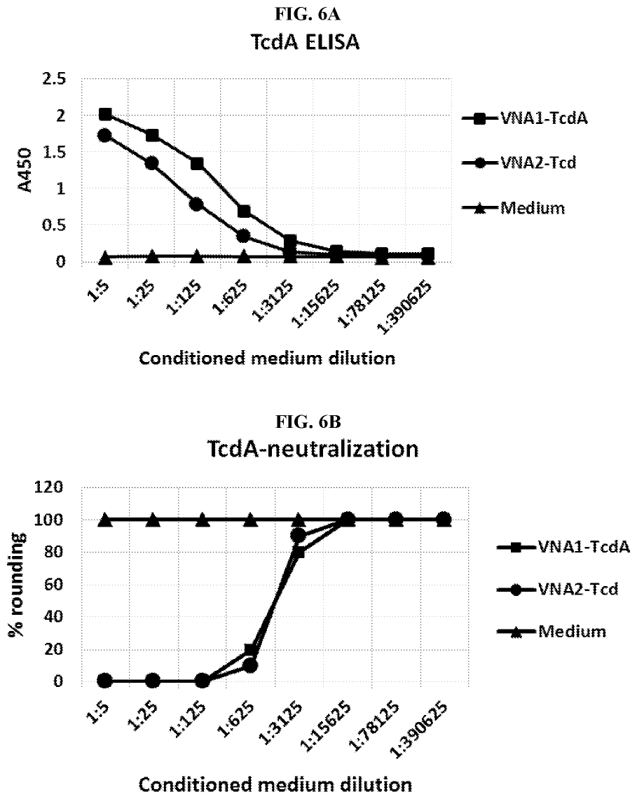

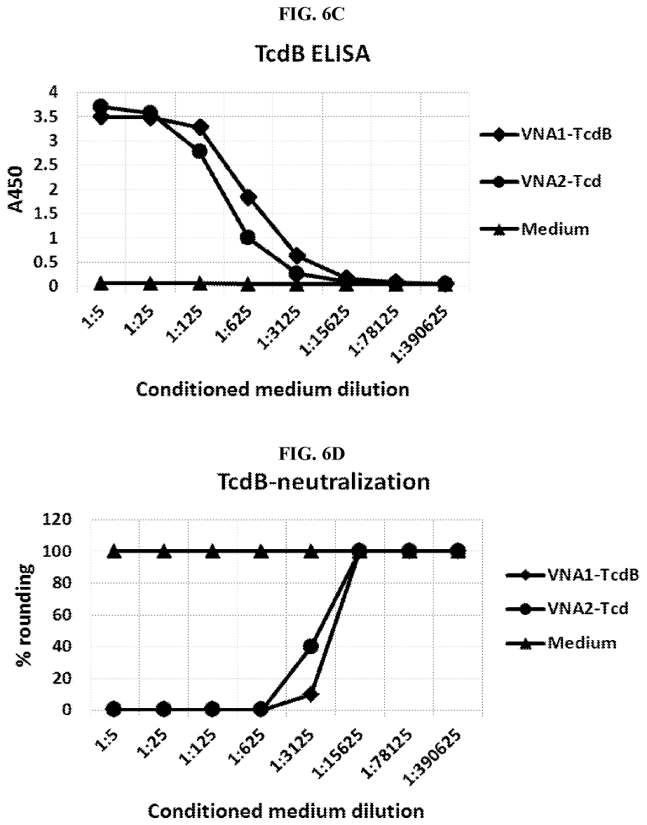

The results obtained following assessment of binding of the VNA-1 TcdA, VNA-1 TcdB and VNA-2 TcdA and TcdB binding molecules described herein by ELISA are shown in FIG. 6A for VNA1-TcdA and VNA2-Tcd (C. difficile toxin A binding molecules) and in FIG. 6C for VNA1-TcdB and VNA2-Tcd (C. difficile toxin B binding molecules). As noted, supernatants from transfected BHK cells were tested by standard dilution ELISA methods using ELISA plates coated with either TcdA or TcdB toxins and by serially diluting the supernatants 1:5. Binding of VNAs to toxin-coated wells was detected using anti-O-tag antibody detection with peroxidase labeled antibodies and assessed by ELISA reader at A450 nm. Medium alone showed no binding of the toxins, while conditioned medium from the transfected cells that expressed the different VNAs clearly showed binding to the appropriate toxins down to a dilution of greater than 1:1000 (FIGS. 6A and 6C).

### Example 3

Specific Neutralization of Toxins by VNA Binding Molecules Targeting C. difficile Toxins A, B, or A and B

To assess the ability of the VNA binding molecules, such as the VNA binding molecules of FIGS. 1-3 herein, to neutralize the toxicity of specific toxins on cells, BHK cells were transfected with nanoparticles comprising RNA encoding the binding molecules. The supernatants were collected at 48 hours post-transfection. The collected supernatants were tested by standard toxin-neutralization methods by performing 1:5 serial dilutions of the supernatants and adding them to 96 well tissue culture plates in which Vero cells were cultured, and then treated with either TcdA or TcdB at known toxic levels. After 24 hours, the plates were assessed for toxicity of the cells by quantifying the percent (%) of cells that had become rounded in each of the wells. Rounded cell morphology indicates cell intoxication by the C. difficile toxins.

As shown in FIGS. 6B and 6D, medium alone did not protect cells from toxin and all wells were 100% rounded. By contrast, conditioned medium from VNA-transfected samples fully protected cells against the appropriate toxin, even when the medium was diluted >100 fold. Partial protection against intoxication of the cells was even seen at 1:1000 or higher dilutions of the TcdA, TcdB, or TcdA and TcdB conditioned medium. (FIGS. 6B and 6D).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A heteromultimeric binding molecule for neutralizing a C. difficile toxin A protein target disease agent, said binding molecule comprising:
a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds;
two C. difficile toxin protein binding components which specifically bind to non-overlapping portions of the C. difficile toxin A, or fragments thereof that retain toxin A activity;
wherein the first epitope tag domain is operably linked to a first C. difficile toxin A binding component comprising the amino acid sequence SGGGLVQPGGSLRLSCAASGFTLDYSSIGWFRQAPGKEREGVSCISSSGDSTKYADSVKGRF TTSRDNAKNTVYLQMNSLKPDDTAVYYCAAFRATMCGVFPLSPYGKDDWGKGTLVTVSS (SEQ ID NO: 6), and a second C. difficile toxin A binding component comprising the amino acid sequence SGGGLVQPGGSLRLSCAASGFTFSDYVMTWVRQAPGKGPEWIATINTDGSTMRDDSTKGR FTISRDNAKNTLYLQMTSLKPEDTALYYCARGRVISASAIRGAVRGPGTQVTVSS (SEQ ID NO: 7) is operably linked to a second epitope tag binding domain comprising the amino acid sequence DELGPRLMGK as set forth in SEQ ID NO: 1;
wherein the C. difficile toxin A binding components are separated from each other by flexible linkers comprising an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof;
wherein separation of the C. difficile toxin A binding components by flexible linkers allows for specific binding of the C. difficile toxin A binding components to non-overlapping portions of C. difficile toxin A, thereby neutralizing the C. difficile toxin A protein; and
wherein specific binding of the epitope tag domains by an anti-epitope tag antibody facilitates clearance of the binding molecule from a subject following neutralization of the C. difficile toxin A protein.

2. A heteromultimeric binding molecule for neutralizing a C. difficile toxin B protein target disease agent, said binding molecule comprising:
a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds;
two C. difficile toxin protein binding components which specifically bind to non-overlapping portions of the C. difficile toxin B, or fragments thereof that retain toxin B activity;
wherein the first epitope tag domain is operably linked to a first C. difficile toxin B binding component comprising the amino acid sequence SGGGLVQPGGSLRLSCEASGFTLDYYGIGWFRQPPGKEREAVSYISASARTILYADSVKGRF TISRDNAKNAVYLQMNSLKREDTAVYYCARRRFSASSVNRWLADDYDVWGRGTQVAVS S (SEQ ID NO: 11), and a second C. difficile toxin B binding component comprising the amino acid sequence SGGGLVQTGGSLRLSCASSGSIAGFETVTWSRQAPGKSLQWVASMTKTNNEIYSDSVKGRF IISRDNAKNTVYLQMNSLKPEDTGVYFCKGPELRGQGIQVTVSS (SEQ ID NO: 12) is operably linked to a second epitope tag binding domain comprising the amino acid sequence DELGPRLMGK as set forth in SEQ ID NO: 1;
wherein the C. difficile toxin B binding components are separated from each other by flexible linkers comprising an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof;
wherein separation of the C. difficile toxin B binding components by flexible linkers allows for specific binding of the C. difficile toxin B binding components to non-overlapping portions of C. difficile toxin B, thereby neutralizing the C. difficile toxin B protein; and
wherein specific binding of the epitope tag domains by an anti-epitope tag antibody facilitates clearance of the binding molecule from a subject following neutralization of the C. difficile toxin B protein.

3. A heteromultimeric binding molecule for neutralizing C. difficile toxin A and toxin B protein target disease agents, said binding molecule comprising:
a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds;
four C. difficile toxin protein binding components, two of which specifically bind to non-overlapping portions of C. difficile toxin A, or fragments thereof that retain toxin A activity and two of which specifically bind to non-overlapping portions of C. difficile toxin B, or fragments thereof that retain toxin B activity;
wherein a first C. difficile toxin A binding component comprises the amino acid sequence SGGGLVQPGGSLRLSCAASGFTLDYSSIGWFRQAPGKEREGVSCISSSGDSTKYADSVKGRF TTSRDNAKNTVYLQMNSLKPDDTAVYYCAAFRATMCGVFPLSPYGKDDWGKGTLVTVSS (SEQ ID NO: 6), and a second C. difficile toxin A binding component comprises the amino acid sequence SGGGLVQPGGSLRLSCAASGFTFSDYVMTWVRQAPGKGPEWIATINTDGSTMRDDSTKGR FTISRDNAKNTLYLQMTSLKPEDTALYYCARGRVISASAIRGAVRGPGTQVTVSS (SEQ ID NO: 7), and wherein a first C. difficile toxin B binding component comprises the amino acid sequence SGGGLVQPGGSLRLSCEASGFTLDYYGIGWFRQPPGKEREAVSYISASARTILYADSVKGRF TISRDNAKNAVYLQMNSLKREDTAVYYCARRRFSASSVNRWLADDYDVWGRGTQVAVS S (SEQ ID NO: 11), and a second C. difficile toxin B binding component comprises the amino acid sequence
(SEQ ID NO: 12)
SGGGLVQTGGSLRLSCASSGSIAGFETVTWSRQAPGKSLQWVASMTKTNN
EIYSDSVKGRFIISRDNAKNTVYLQMNSLKPEDTGVYFCKGPELRGQGIQ
VTVSS;
wherein one terminus of the first C. difficile toxin A binding component is flexibly linked to one terminus of the first C. difficile toxin B binding component; a second terminus of the first C. difficile toxin B binding component is flexibly linked to one terminus of the second C. difficile toxin B binding component; a second terminus of the second C. difficile toxin B binding component is flexibly linked to one terminus of the second C. difficile toxin A binding component; and a second terminus of the second C. difficile toxin A binding component is flexibly linked to a second epitope tag binding domain comprising the amino acid sequence DELGPRLMGK as set forth in SEQ ID NO: 1;
wherein the linker comprises an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof;
wherein separation of the C. difficile toxin binding components by flexible linkers allows for specific binding of the C. difficile toxin binding components to non-overlapping portions of C. difficile toxin A and toxin B, thereby neutralizing the C. difficile toxin A and toxin B proteins; and
wherein specific binding of the epitope tag domains by one or more an anti-epitope tag antibodies facilitates clearance of the binding molecule from a subject following neutralization of C. difficile toxin A and toxin B.

4. A heteromultimeric binding molecule for neutralizing a C. botulinum toxin A protein target disease agent, said binding molecule comprising:
a cleavable secretory domain at one terminus of the binding molecule, said domain allowing for secretion of the binding molecule from cells; wherein the cleavable secretory domain is operably linked to a first epitope tag domain comprising the amino acid sequence DELGPRLMGK as set forth in SEQ ID NO: 1 to which an anti-epitope tag antibody specifically binds;
two C. botulinum toxin protein binding components which specifically bind to non-overlapping portions of the C. botulinum toxin A protein, or fragments thereof that retain toxin A activity;
wherein the first epitope tag domain is operably linked to a first C. botulinum toxin A binding component comprising the amino acid sequence SGGGLVQVGGSLRLSCVVSGSDISGIAMGWYRQAPGKRREMVADIFSGGSTDYAGSVKGR FTISRDNAKKTSYLQMNNVKPEDTGVYYCRLYGSGDYWGQGTQVTVSS (SEQ ID NO: 13),
and a second C. botulinum toxin A binding component comprising the amino acid sequence SGGGLVHPGGSLRLSCAPSASLPSTPFNPFNNMVGWYRQAPGKQREMVASIGLRINYADSV KGRFTISRDNAKNTVDLQMDSLRPEDSATYYCHIEYTHYWGKGTLVTVSS (SEQ ID NO: 14) is operably linked to a second epitope tag binding domain comprising the amino acid sequence DELGPRLMGK as set forth in SEQ ID NO: 1;
wherein the C. botulinum toxin A binding components are separated from each other by flexible linkers comprising an amino acid sequence selected from the group consisting of QGVQSQLQLVE (SEQ ID NO: 8), EPKTPKPQGGGGSGGGGSGGGGSQGVQSQVQLVE (SEQ ID NO: 9), EPKTPKPQ (SEQ ID NO: 10) and a combination thereof;
wherein separation of the C. botulinum toxin A binding components by flexible linkers allows for specific binding of the C. botulinum toxin A binding components to non-overlapping portions of C. botulinum toxin A, thereby neutralizing the C. botulinum toxin A protein; and
wherein specific binding of the epitope tag domains by an anti-epitope tag antibody facilitates clearance of the binding molecule from a subject following neutralization of the C. botulinum toxin A protein.

5.-6. (canceled)

7. A heteromultimeric binding molecule; wherein the binding molecule comprises two VHH binding regions that specifically bind to and neutralize C. difficile toxin A, wherein the binding molecule has the amino acid sequence set forth in SEQ ID NO: 16; or wherein the binding molecule comprises two VHH binding regions that specifically bind to and neutralize C. difficile toxin B, wherein the binding molecule has the amino acid sequence set forth in SEQ ID NO: 17; or wherein the binding molecule comprises two VHH binding regions that specifically bind to and neutralize C. difficile toxin A and two VHH binding regions that specifically bind to and neutralize C. difficile toxin B, wherein the binding molecule has the amino acid sequence set forth in SEQ ID NO: 18; or wherein the binding molecule comprises two VHH binding regions that specifically bind to and neutralize C. botulinum toxin A, wherein the binding molecule has the amino acid sequence set forth in SEQ ID NO: 19.

8.-10. (canceled)

11. A pharmaceutical composition comprising the binding molecule of claim 2 and a pharmaceutically acceptable carrier, excipient, or vehicle.

12. A polynucleotide encoding the binding molecule of claim 2.

13. The polynucleotide according to claim 12, which is an RNA molecule, a DNA molecule, a messenger RNA (mRNA) molecule, or a mRNA molecule comprising a codon optimized sequence for human expression.

14.-16. (canceled)

17. An expression vector comprising the polynucleotide according to claim 12, and which comprises an operably linked promoter sequence, signal sequence and stop codon sequence.

18.-19. (canceled)

20. A host cell comprising the expression vector according to claim 17.

21.-22. (canceled)

23. A lipid nanoparticle comprising the mRNA polynucleotide according to claim 13.

24. (canceled)

25. A method of neutralizing a target disease agent or a toxin, said method comprising contacting the target disease agent or the toxin with the heteromultimeric binding molecule according to claim 2, in an amount effective for the binding molecule to bind to and neutralize the target disease agent or the toxin.

26. A method of neutralizing a target disease agent or a toxin, said method comprising contacting the target disease agent or the toxin with the pharmaceutical composition according to claim 11, in an amount effective for the binding molecule to bind to and neutralize the target disease agent or the toxin.

27. The method according to claim 25, wherein the target disease agent or toxin is neutralized in vitro or in vivo.

28. (canceled)

29. A method of treating or preventing intoxication of a subject by a toxin, said method comprising: administering to a subject in need thereof the heteromultimeric binding molecule according to claim 2, in an effective amount for the binding molecule to neutralize the toxin in the subject after intoxication or prior to the subject's having symptoms of intoxication.

30. A method of treating or preventing intoxication of a subject by a toxin, said method comprising: administering to a subject in need thereof the pharmaceutical composition according to claim 11, in an effective amount for the binding molecule to neutralize the toxin in the subject after intoxication or prior to the subject's having symptoms of intoxication.

31. A method of neutralizing a target disease agent or a toxin, said method comprising contacting the target disease agent or the toxin with the polynucleotide according to claim 12, in an amount effective for expression of the binding molecule which binds to and neutralizes the target disease agent or the toxin.

32.-36. (canceled)

37. The heteromultimeric binding molecule according to claim 2, which is recombinantly produced.

38. A kit comprising the heteromultimeric binding molecule according to claim 2 for treating or monitoring a subject exposed to or at risk or exposure to infection by C. difficile or C. botulinum and/or to disease resulting therefrom.

39.-41. (canceled)

42. The method of claim 29, further comprising: administering to the subject an anti-epitope tag antibody or an epitope tag-binding fragment of the anti-epitope tag antibody.

43. The method of claim 30, further comprising: administering to the subject an anti-epitope tag antibody or an epitope tag-binding fragment of the anti-epitope tag antibody.


SEQUENCE LISTING (21 sequences)

SEQ ID NO: 1 - protein, 10 aa, Artificial Sequence
  Description of Artificial Sequence Synthetic peptide
DELGPRLMGK

SEQ ID NO: 2 - protein, 18 aa, Artificial Sequence
  Description of Artificial Sequence Synthetic peptide
GGGGDELGPRLMGKGGGG

SEQ ID NO: 3 - protein, 13 aa, Artificial Sequence
  Description of Artificial Sequence Synthetic peptide
GAPVPYPDPLEPR

SEQ ID NO: 4 - protein, 5 aa, Artificial Sequence
  Description of Artificial Sequence Synthetic peptide
GGGGS

SEQ ID NO: 5 - protein, 15 aa, Artificial Sequence
  Description of Artificial Sequence Synthetic peptide
GGGGSGGGGSGGGGS

SEQ ID NO: 6 - protein, 121 aa, Artificial Sequence
  Description of Artificial Sequence Synthetic polypeptide
SGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSS

SEQ ID NO: 7 - protein, 115 aa, Artificial Sequence
  Description of Artificial Sequence Synthetic polypeptide
SGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSS

SEQ ID NO: 8 - protein, 11 aa, Artificial Sequence
  Description of Artificial Sequence Synthetic peptide
QGVQSQLQLVE

SEQ ID NO: 9 - protein, 34 aa, Artificial Sequence
  Description of Artificial Sequence Synthetic polypeptide
EPKTPKPQGGGGSGGGGSGGGGSQGVQSQVQLVE

SEQ ID NO: 10 - protein, 8 aa, Artificial Sequence
  Description of Artificial Sequence Synthetic peptide
EPKTPKPQ

SEQ ID NO: 11 - protein, 121 aa, Artificial Sequence
  Description of Artificial Sequence Synthetic polypeptide
SGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSS

SEQ ID NO: 12 - protein, 105 aa, Artificial Sequence
  Description of Artificial Sequence Synthetic polypeptide
SGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSS

SEQ ID NO: 13 - protein, 108 aa, Artificial Sequence
  Description of Artificial Sequence Synthetic polypeptide
SGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSS

SEQ ID NO: 14 - protein, 111 aa, Artificial Sequence
  Description of Artificial Sequence Synthetic polypeptide
SGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSS

SEQ ID NO: 15 - protein, 13 aa, Artificial Sequence
  Description of Artificial Sequence Synthetic peptide
DICLPRWGCLWED

SEQ ID NO: 16 - protein, 374 aa, Artificial Sequence
  Description of Artificial Sequence Synthetic polypeptide
METDTLLLWVLLLWVPGSTGDAAQPARRARRTKLSGGGGDELGPRLMGKGGGGQGVQSQLQLVESGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSEPKTPKPQGGGGSGGGGSGGGGSQGVQSQVQLVESGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSEPKTPKPQGGGGDELGPRLMGKGGGGSDICLPRWGCLWED

SEQ ID NO: 17 - protein, 364 aa, Artificial Sequence
  Description of Artificial Sequence Synthetic polypeptide
METDTLLLWVLLLWVPGSTGDAAQPARRARRTKLSGGGGDELGPRLMGKGGGGQGVQSQLQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSEPKTPKPQGGGGSGGGGSGGGGSQGVQSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQGGGGDELGPRLMGKGGGGSDICLPRWGCLWED

SEQ ID NO: 18 - protein, 667 aa, Artificial Sequence
  Description of Artificial Sequence Synthetic polypeptide
METDTLLLWVLLLWVPGSTGDAAQPARRARRTKLSGGGGDELGPRLMGKGGGGQGVQSQLQLVESGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSEPKTPKPQGGGGSGGGGSGGGGQGVQSQLQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSEPKTPKPQGGGGSGGGGSGGGGSQGVQSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQGGGGSGGGGSGGGGSQGVQSQVQLVESGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSEPKTPKPQGGGGDELGPRLMGKGGGGSDICLPRWGCLWED

SEQ ID NO: 19 - protein, 360 aa, Artificial Sequence
  Description of Artificial Sequence Synthetic polypeptide
METDTLLLWVLLLWVPGSTGDAAQPARRARRTKLSGGGGDELGPRLMGKGGGGQGVQAQLQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPSGGGGSGGGGSGGGGSGGQGVQAQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQSGGGGDELGPRLMGKGGGGSDICLPRWGCLWED

SEQ ID NO: 20 - protein, 457 aa, Artificial Sequence
  Description of Artificial Sequence Synthetic polypeptide
MNFGLSLIFLALILKGVQCEVQLVETGGGLVQPGRSLKLTCAASGFTFSAAWMHWVRQSPNKRLDWVARIKDKSNNYATDYVESVKGRFTISRDDSKSSLYLQMNNLKEEDSATYYCVTSEDWGQGVMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWPNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 21 - protein, 234 aa, Artificial Sequence
  Description of Artificial Sequence Synthetic polypeptide
MESQTQLLGLLLLWVPGMRCDIQMTQSPSFLSASVGDRATFNCKASQNIARSLNWYQQKLGEAPKLLIYNTNTLQPGIPSRFSGSGSGTDFTLTISSLQPEDVATFFCLQFGSWPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC